(19) United States
(12) Patent Application Publication
(10) Pub. No.: US 2021/0189002 A1
(43) Pub. Date: Jun. 24, 2021

(54) RECOMBINANT ORGANISMS AND METHODS FOR PRODUCING GLYCOMOLECULES WITH HIGH GLYCAN OCCUPANCY

(71) Applicant: Conagen Inc., Bedford, MA (US)

(72) Inventors: Nicky C. Caiazza, Rancho Santa Fe, CA (US); Jun Urano, Irvine, CA (US); Spiros Kambourakis, Rancho Santa Fe, CA (US)

(73) Assignee: Conagen Inc., Bedford, MA (US)

(21) Appl. No.: 17/084,929

(22) Filed: Oct. 30, 2020

Related U.S. Application Data
(63) Continuation of application No. PCT/US2019/029936, filed on Apr. 30, 2019.
(60) Provisional application No. 62/665,270, filed on May 1, 2018.

(51) Int. Cl. C07K 16/32; C12N 9/10

(52) U.S. Cl. CPC C07K 16/32; C07K 2317/14; C07K 2317/24; C12N 9/1051

(57) ABSTRACT

The invention provides a recombinant Thraustochytriaceae cell for the production of a glycomolecule. The cell comprises a nucleic acid encoding a heterologous glycomolecule, and a sequence encoding a heterologous oligosaccharyltransferase. The cell produces the heterologous glycomolecule having a higher glycan occupancy compared to the same heterologous glycomolecule produced by a corresponding cell not comprising the heterologous oligosaccharyltransferase. The glycan occupancy can be more than 25%. The cells advantageously produce and, optionally secrete, the heterologous glycomolecule. Thus, the invention provides recombinant organisms that provide glycomolecules having a glycosylation profile that is more similar to the glycosylation profile produced in a manunalian cell.

### CROSS-REFERENCE TO RELATED APPLICATIONS

This application claims benefit of priority under 35 U.S.C. § 119(e) of U.S. Ser. No. 62/665,270, filed May 1, 2018, the entire contents of which is incorporated herein by reference in its entirety.

The material in the accompanying sequence listing is hereby incorporated by reference into this application. The accompanying sequence listing text file, name SGI2180 1WO Sequence Listing.txt was created on Apr. 23, 2019, and is 111 kb. The file can be accessed using Microsoft Word on a computer that uses Windows OS.

### FIELD OF THE INVENTION

The invention relates to recombinant organisms and methods for producing glycomolecules having high glycan occupancy.

### INCORPORATION OF SEQUENCE LISTING

The material in the accompanying sequence listing is hereby incorporated by reference into this application. The accompanying sequence listing text file, name SGI2180 Sequence Listing.txt, was created on May 1, 2017, and is 111 kb. The file can be assessed using Microsoft Word on a computer that uses Windows OS.

### BACKGROUND OF THE INVENTION

Glycomolecules include drugs that are an important therapeutic resource for the treatment of a variety of diseases and disorders. This class of drugs includes monoclonal antibodies, which are very useful in many applications, Many glycomolecule drugs require glycosylation for optimal efficacy in humans and animals. However, different types of host cells (e.g. mammals, plants, insects, fungi, etc.) produce different glycosylation profiles. This therefore presents concerns as the glycosylation profile produced on a therapeutic glycomolecule produced in non-mammalian host cells could elicit an immunogenic response in a human or animal patient treated with the therapeutic. Furthermore, some genetic modifications to host cells result in the cell producing a heterologous protein or peptide that lacks glycosylation at site where it is desirably present.

Therapeutic glycomolecules are often produced in yeasts and fungi. While some engineering in these cell types has been performed to cause these organisms to produce more mammalian-like glycosylation profiles, these organisms are slow growing. Furthermore, some genetic engineering performed on these host cells results in a lack of glycosylation at sites where it is desirable to be present. It would therefore be of great advantage to have host cell systems that grow quickly and are able to produce therapeutic glycomolecules having N-linked glycosylation profiles similar to what is produced by mammalian cells, including appropriate site glycosylation.

### SUMMARY OF THE INVENTION

The invention provides recombinant host cells or organisms containing a nucleic acid encoding a heterologous glycomolecule, which is produced by the cell or organism. The glycomolecule can have a high level of glycan occupancy. In one embodiment the heterologous glycomolecule is an immunoglobulin molecule. The recombinant host cells express and produce one or more oligosaccharyl transferase (OST) gene(s), which can be heterologous OST gene(s). The host cells can also contain a genetic modification to one or more genes encoding a mannosyl transferase. The cells can advantageously produce and, optionally, secrete the heterologous glycomolecule, which has higher glycan occupancy than the same heterologous glycomolecule produced by a corresponding cell that does not comprise produce or express the one or more OSTs. The glycomolecule produced can therefore have a glycosylation profile that is more similar to the glycosylation profile for the same glycomolecule produced in a mammalian cell, and therefore be safer or more effective for use as a therapeutic in humans or animals. In various embodiments the glycomolecule can be a glycoprotein, glycopeptide, or glycolipid.

In a first aspect the invention provides recombinant cells of the family Thraustochytriaceae for the production of a glycomolecule. The recombinant cell can have a nucleic acid encoding of a heterologous glycomolecule, and a sequence encoding a heterologous oligosaccharyltransferase. The recombinant cells can produce the heterologous glycomolecule, having higher glycan occupancy compared to the same heterologous glycomolecule produced by a corresponding cell not comprising the heterologous oligosaccharyltransferase. In some embodiments the glycomolecule is a glycoprotein or glycopeptide. The recombinant cell can optionally have a genetic modification in a mannosyl transferase gene, and the mannosyl transferase gene can be alg3.

In some embodiments the heterologous oligosaccharyltransferase is from a protozoa, and can also have a protozoan promoter that regulates the sequence encoding the heterologous oligosaccharyltransferase. The heterologous oligosaccharyltransferase can be a single protein enzyme. In some embodiments the oligosaccharyltransferase (OST) is from a protozoa of the Family Trypanosomatidae, for example a trypanosome, and can also be an OST from an organism of the genus Leishmania. The heterologous OST can be the Stt3 subunit of a protozoan OST.

In some embodiments the heterologous OST is a protozoan enzyme encoded by a gene selected from the group TbStt3A, TbStt3B, LmStt3D,Stt3 1, and LbStt3 3. In some embodiments the protozoan gene is under the control of a promoter from an organism of the family Thraustochytriaceae.

In various embodiments the heterologous glycoprotein or glycopeptide produced by the recombinant cell of the invention can produce a glycan profile having more than 25% glycan occupancy, or more than 50% glycan occupancy. The recombinant cell can produce and secrete the heterologous glycoprotein or glycopeptide molecule or a functional portion thereof. The heterologous glycoprotein or glycopeptide can be an antibody molecule, or functional portion thereof. The glycan profile can have N-glycans, and can comprise Man3GlcNAc2 and/or Man4GlcNAc2, and/or Man5GlcNAc2, or any combination or sub-combination of them.

In various embodiments the recombinant cell can be of the family Thraustochytriaceae, and can be from a genus selected from the group Japanochytrium, Oblongichytrium, Thraustochytrium, Aurantiochytrium, and Schizochytrium.

In various embodiments heterologous glycoprotein or glycopeptide can be any of trastuzumab, eculizumab, natalizumab, cetuximab, omalizumab, usteinumab, paniturnumab, or adalimumab, or a functional fragment of any of them.

In another aspect the invention provides a composition comprising any of the heterologous glycoproteins or glycopeptides produced by the recombinant cells described herein. The composition can be provided in a pharmaceutically acceptable carrier.

In another aspect the invention provides a method of producing a glycomolecule having high glycan occupancy. The method can involve steps of providing a recombinant Thraustochytriaceae cell having a nucleic acid encoding a heterologous glycomolecule, and a sequence encoding a heterologous oligosaccharyltransferase. The recombinant cell can produce the heterologous glycomolecule having higher gly can occupancy compared to the same heterologous glycomolecule produced by a corresponding cell not comprising the heterologous oligosaccharyltransferase. The recombinant cell can be any described herein, and can have any of the features of any cell described herein.

In another aspect the invention provides a recombinant cell of the family Thraustochytriaceae for the production of a glycomolecule. The recombinant cell has a nucleic acid sequence encoding a heterologous glycomolecule, and a nucleic acid sequence encoding a promoter that overexpresses an endogenous oligosaccharyltransferase, wherein the recombinant cell produces the heterologous glycomolecule having higher glycan occupancy compared to the same heterologous glycomolecule produced by a corresponding cell not comprising the heterologous oligosaccharyltransferase.

The summary of the invention described above is not limiting and other features and advantages of the invention will be apparent from the following detailed description of the invention, and from the claims.

### DESCRIPTION OF THE DRAWINGS

FIG. 1 provides a bar graph showing that the expression of OSTs in Thraustochytriaceae cells of the invention result in an increase in glycan occupancy on the heterologous glycoprotein also expressed in the cell. The bars show the percentage of glycosylation sites on trastuzumab occupied by an N-glycan. Parent cell 18.7%; ChStt3 45.5%: LbStt3 3 62.9%.

FIGS. 2A-2E; FIG. 2a provides a map of construct pCAB056. FIG. 2b provides a map of construct pCAB-057. FIG. 2c provides a map of construct pCAB-060. FIG. 2d provides a map of construct pCAB-061. FIG. 2e provides a map of construct pSGI-AM-001.

FIGS. 3A-3C; FIG. 3a shows an illustration of a structure of Man3GlcNAc2 with fucose attached; FIG. 3b shows an illustration of a structure of Man5GlcNAc2. FIG. 3c provides an illustration of a Man5GlcNAc2 glycan structure.

FIG. 4 shows an illustration of various glycan structures from various species. It is seen that human and animal glycan structures have a Man3 core structure, while the yeast glycans have a high glycan core structure.

### DESCRIPTION OF THE INVENTION

The invention provides recombinant cells or organisms that contain a nucleic acid molecule encoding the amino acid sequence of a heterologous glycomolecule. The organisms can also express a heterologous oligosaccharyl transferase (OST) enzyme. In some embodiments the organisms can overexpress an endogenous OST enzyme. The organisms produce the heterologous glycoprotein or glycopeptide with a glycosylation profile that contains a higher amount of glycosylated sites on their polypeptide chains compared to the same glycoprotein or glycopeptide produced by a corresponding organism or host cell that does not express the heterologous OST (or overexpress an endogenous OST) and that is cultivated under the same conditions. In any of the embodiments disclosed herein the recombinant cells of the invention also have a genetic modification to one or more mannosyl transferase genes.

Human and animal therapeutic molecules that are glycomolecules that contain sites on the molecule that are occupied by a glycan moiety in their natural, safer, or more effective form. These molecules can be produced as heterologous glycoproteins or glycopepetides having the preferred or full glycan occupancy by a mammalian cell, e.g. a CHO cell. A level or amount of glycan occupancy similar to or the same as the heterologous glycomolecule produced by a human or another mammalian cell is therefore desirable. The level or extent of glycans occupying these sites is referred to as glycan occupancy. But in non-mammalian cell types the amount of glycans occupying sites that contain a glycan when the same heterologous glycomolecule is produced by a mammalian cell can be low, with sites that are desirably occupied by a glycan moiety instead missing their glycan moiety, or it otherwise being absent. The present inventors discovered unexpectedly that expression of one or more heterologous OSTs (or overexpression of an endogenous OST) in the cells or organisms of the invention results in an increase in glycan occupancy in the heterologous glycoprotein or glycopeptide produced by the cell. The increase in glycan occupancy can restore a glycan occupancy level lost due to properties of the cell or that occurred due to other genetic modifications made to the cell. The produced glycoprotein or glycopeptide produced by the cells of the invention therefore have significantly more glycan moieties occupying glycosylation sites on the polypeptide chain as a result of the expression or overexpression of the one or more OSTs. The discovery therefore allows for the production of glycomolecules in cells of the invention having higher glycan occupancy. Therefore, the glycomolecule may be safer, more stable, or more effective for use as a therapeutic molecule, and/or less likely to provoke an immune response in a human or other mammal. In any of the embodiments disclosed herein the glycomolecule can be a glycoprotein, glycopeptide, or glycolipid.

Glycosylation is a common co-translational modification in eukaryotic or mammalian cells. It generally involves the transfer of a lipid-linked oligosaccharide onto asparagine residues in a tripeptide sequon on a nascent protein, which can occur in the lumen of the endoplasmic reticulum. The attachment of an oligosaccharide unit to the polypeptide at the site of occupancy can enhance solubility, improve folding, facilitate secretion, modulate antigenicity, and increase in vivo half-life of the glycoprotein.

Many proteins, peptides, and lipids produced by living organisms are modified by glycosylation. Glycoproteins and glycopeptides are proteins or peptides that have carbohydrate groups covalently attached to their polypeptide chain; glycolipids are lipid molecules with a carbohydrate attached by a glycosidic bond. In various embodiments the glycoproteins or glycopeptides can have at least one carbohydrate moiety attached to the polypeptide chain at two or at least two, or 2-3 or 2-4 or 2-5 or at least three or at least four or at least five or at least six or at least seven or at least eight or at least nine, or at least ten carbohydrate moieties attached to at least one polypeptide chain of the glycoprotein, glycopeptide, or glycolipid.

The glycan profile can indicate the types of glycans present in a molecule, their composition and structure, including the percentage or amount of glycans in the profile that are sulfated or unsulfated. The glycan (or glycosylation) profile of the glycomolecules can be important for various reasons, such as cellular recognition signals, to prevent an immune response against the protein or peptide, for protein folding, and for stability. Glycosylation can occur to produce any one or more of N-linked glycans, O-linked glycans, C-linked glycans, or phosphoglycosylation, or any combination or sub-combination thereof. N-linked glycosylation refers to the attachment of a sugar molecule (or oligosaccharide known as glycan) to a nitrogen atom, for example an amide nitrogen of asparagine, in the sequence of a protein or peptide. An N-linked glycan (or N-glycan) profile refers to the specific glycosylation (e.g. mono- or oligosaccharide) patterns present on a particular glycomolecule, or group of glycoproteins, glycopeptides, or glycolipids at such nitrogen atoms. The N-glycan profile of a glycomolecule can be a description of the number and structure of N-linked mono- or oligosaccharides that are associated with the particular glycomolecule. O-linked glycosylation refers to the attachment of a sugar molecule to an oxygen atom in an amino acid of a protein or peptide (e.g. serine or threonine). C-linked glycosylation can occur when mannose binds to the indole ring of tryptophan. Phosphoglycosylation occurs when a glycan binds to serine via the phosphodiester bond.

Monoclonal antibodies and other immunoglobulins are just two of many categories of glycoproteins that the invention can be applied to. In some embodiments the N-linked glycans of an N-glycan profile can be attached to the nitrogen atom of an asparagine sidechain that can be present as part of the consensus peptide sequence Asn-X-Thr/Ser of a glycomolecule, where X is any amino acid except proline and Thr/Ser is either threonine or serine.

### Host Cells

In some embodiments the recombinant cells or organisms of the invention are from the Class Labyrinthulomycetes. The Labyrinthulomycetes are single-celled marine decomposers that generally consume non-living plant, algal, and animal matter. They are ubiquitous and abundant, particularly on dead vegetation and in salt marshes and mangrove swamps. While the classification of the Thraustochytrids and Labyrinthulids has evolved over the years, for the purposes of the present application, “Labyrinthulomycetes” is a comprehensive term that includes microorganisms of the Orders Thraustochytriales and Labyrinthulid. Organisms of the Order Thraustochytriales and Order Labyrinthulid are useful in the invention and include (without limitation) the genera Althornia, Aplanochytrium, Aurantiochytrium, Botyrochytrium, Corallochytrium, Diplophryids, Diplophrys, Elina, Japonochytrium, Labyrinthula, Labryinthuloides, Oblongichytrium, Pyrrhosorus, Parietichytrium, Sicyoidochytrium, Schizochytrium, Thraustochytrium, and Ulkenia. The recombinant host cells of the invention can also be a member of the Order Labyrinthulales.

In some embodiments the host cell or organism of the invention can be an organism of the Class Labyrinthulomycetes and the taxonomic family Thraustochytriaceae, which family includes but is not limited to any one or more of the genera Thraustochytrium, Japonochytrium, Aurantiochytrium, Aplanochytrium, Sycyoidochytrium, Botryochytrium, Parietichytrium, Oblongochytrium, Parietichytrium, Schizochytrium, Ulkenia, and Elina, or any group comprising a combination or sub-combination of them, which is disclosed as if set forth fully herein in all possible combinations. Examples of suitable microbial species of the invention within the genera include, but are not limited to: any Schizochytrium species, including, but not limited to, Schizochytrium aggregatum, Schizochytrium limacinum, Schizochytrium minutum, Schizochytrium mangrovei, Schizochytrium marinum, Schizochytrium octosporum, and any Aurantiochytrium sp., any Thraustochytrium species (including former Ulkenia species such as U. visurgensis, U. amoeboida, U. sarkariana, U. profunda, U. radiata, U. minuta and Ulkenia sp. BP-5601), and including Thraustochytrium striatum, Thraustochytrium aureum, Thraustochytrium roseum; and any Japonochytrium sp. Strains of Thraustochytriales that may be particularly suitable for the presently disclosed invention include, but are not limited to: Schizochytrium sp. (S31) (ATCC 20888); Schizochytrium sp. (S8) (ATCC 20889); Schizochytrium sp. (LC-RM) (ATCC 18915); Schizochytrium sp. (SR21); Schizochytrium aggregatum (ATCC 28209); Schizochytrium limacinum (IFO 32693); Thraustochytrium sp. 23B ATCC 20891; Thraustochytrium striatum ATCC 24473; Thraustochytrium aureum ATCC 34304); Thraustochytrium roseum (ATCC 28210; and Japonochytrium sp. LI ATCC 28207. In some embodiments the recombinant host cell of the invention can be selected from an Aurantiochytrium or a Schizochytrium or a Thraustochytrium, or all of the three groups together or any combination or sub-combination of them. The recombinant host cell of the invention can be selected from any combination of the above taxonomic groups, which are hereby disclosed as every possible combination or sub-combination as if set forth fully herein.

The cells or organisms of the invention can be recombinant, which are cells or organisms that contain a recombinant nucleic acid. The recombinant nucleic acid can encode a functional glycomolecule that is expressed in and, optionally, secreted from the recombinant cell. The term “recombinant” nucleic acid molecule as used herein, refers to a nucleic acid molecule that has been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As nonlimiting examples, a recombinant nucleic acid molecule can include any of: 1) a nucleic acid molecule that has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination)) of nucleic acid molecules; 2) include conjoined nucleotide sequences that are not conjoined in nature, 3) has been engineered using molecular cloning techniques such that it lacks one or more nucleotides with respect to the naturally occurring nucleic acid molecule sequence, and/or 4) has been manipulated using molecular cloning techniques such that it has one or more sequence changes or rearrangements with respect to the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. A recombinant cell contains a recombinant nucleic acid.

As used herein, “exogenous” with respect to a nucleic acid or gene indicates that the nucleic acid or gene has been introduced (e.g. “transformed”) into an organism, microorganism, or cell by human intervention. Typically, such an exogenous nucleic acid is introduced into a cell or organism via a recombinant nucleic acid construct. An exogenous nucleic acid can be a sequence from one species introduced into another species, i.e., a heterologous nucleic acid. A heterologous nucleic acid can also be an exogenous synthetic sequence not found in the species into which it is introduced. An exogenous nucleic acid can also be a sequence that is homologous to an organism (i.e., the nucleic acid sequence occurs naturally in that species or encodes a polypeptide that occurs naturally in the host species) that has been isolated and subsequently reintroduced into cells of that organism. An exogenous nucleic acid that includes a homologous sequence can often be distinguished from the naturally-occurring sequence by the presence of non-natural sequences linked to the exogenous nucleic acid, e.g., non-native regulatory sequences flanking the homologous gene sequence in a recombinant nucleic acid construct. Alternatively or in addition, a stably transformed exogenous nucleic acid can be detected and/or distinguished from a native gene by its juxtaposition to sequences in the genome where it has integrated. Further, a nucleic acid is considered exogenous if it has been introduced into a progenitor of the cell, organism, or strain under consideration.

When applied to organisms, the terms “transgenic” “transformed” or “recombinant” or “engineered” or “genetically engineered” refer to organisms that have been manipulated by introduction of an exogenous or recombinant nucleic acid sequence into the organism, or by the manipulation of native sequences, which are therefore then recombinant (e.g. by mutation of sequences, deletions, insertions, replacements, and other manipulations described below). In some embodiments the exogenous or recombinant nucleic acid can express a heterologous protein product. Non-limiting examples of such manipulations include gene knockouts, targeted mutations and gene replacement, gene replacement, promoter replacement, deletions or insertions, disruptions in a gene or regulatory sequence, as well as introduction of transgenes into the organism. For example, a transgenic microorganism can include an introduced exogenous regulatory sequence operably linked to an endogenous gene of the transgenic microorganism. Recombinant or genetically engineered organisms can also be organisms into which constructs for gene “knock down,” deletion, or disruption have been introduced. Such constructs include, but are not limited to, RNAi, microRNA, shRNA, antisense, and ribozyme constructs. Also included are organisms whose genomes have been altered by the activity of meganucleases or zinc finger nucleases. A heterologous or recombinant nucleic acid molecule can be integrated into a genetically engineered/recombinant organism's genome or, in other instances, not integrated into a recombinant/genetically engineered organism's genome, or on a vector or other nucleic acid construct. As used herein, “recombinant microorganism” or “recombinant host cell” includes progeny or derivatives of the recombinant microorganisms of the disclosure. Because certain modifications may occur in succeeding generations from either mutation or environmental influences, such progeny or derivatives may not, in fact, be identical to the parent cell, but are still included within the scope of the term as used herein.

The host cells of the invention described herein carry important advantages over other cell types. The host cells or organisms of the invention require only a genetic modification (e.g. a deletion or disruption) to one or more mannosyl transferase gene(s) (e.g. alg3) to produce a heterologous glycomolecule having fewer high mannose structures, and more paucimannose (Man3 and/or Man4) structures compared to the same glycomolecule produced by a corresponding cell not having the genetic modification to one or more alg3 gene(s) and cultivated under the same conditions as described herein. Thus, the Labyrinthulomycetes host cells described herein require only a single deletion of a mannosyl transferase gene to produce a heterologous glycoprotein or glycopeptide having an N-linked glycan profile described herein. Therefore, the invention allows Man3 and/or Man4 (or paucimannose structures) to be produced more efficiently with less effort by selecting a host with greater abilities to produce these structures.

Thus, in any of the embodiments the host cells or organisms of the invention contain a minimum of genetic modifications or genetic manipulations. In any of the embodiments the host cells or organisms of the invention do not comprise a deletion of an alpha-1,6-mannosyltransferase, or contain only wild-type alpha-1,6-mannosyltransferases, which are not overexpressed or genetically modified. The cells do not need, and can have an absence of, genetic modification of protein mannosyltransferase genes (e.g, deletions or disruptions), do not require the presence of Pmtp inhibitors at any point of production of the heterologous glycomolecule, and do not require the presence or use of alpha-1,2-mannosidase or any exogenous mannosidases to reduce mannose moieties on the heterologous glycomolecule produced by the cell; and do not require or have a genetic modification to any beta-mannosyltransferase gene (e.g. deletion or disruption of BMT1, BMT2, BMT3, or BMT4).

In any of the embodiments the host cells or organisms of the invention can contain only a single genetic modification of a gene encoding a mannosyl transferase enzyme. In any of the embodiments the single mannosyl transferase gene modification can be to the alg3 gene. In any of the embodiments all mannosyl transferase enzymes except alg3 can be expressed from wild-type genes encoding the enzymes and present on the genome, e.g. the host cell or organism can express the wild type alg11 gene. In another embodiment the host cells can have a genetic modification to alg3, and alg9 and/or alg12, but no other genetic modifications to any other mannosyl transferase gene.

In any of the embodiments the cells can also not comprise any heterologous enzymes. The host cells or organisms of the invention can contain no heterologous flippases, and/or can contain no heterologous mannosidases and/or no overexpressed homologous or wild-type mannosidases, and additionally can contain no heterologous glycolipid translocation protein, examples including but not limited to Rft1 and/or Rft1p. Also any of the embodiments of the host cells or organisms of the invention can contain no overexpression of wild-type or exogenous flippases or wild-type or exogenous glycolipid translocation protein(s). The host cells also do not have or require the deletion or disruption of the ATT1 (acquired thermotolerance 1) gene; and does not have or require the deletion or disruption of the OCH1 (Outer Chain) gene; and does not have or require the deletion or disruption of an osteosarcoma gene (e.g. OS-9). The host cells can have natural, wild-type genes for all of these genes. The host cells can also not comprise any exogenous or recombinant GnTI or GnTII genes. The host cells can also not have any mutations to reduce or eliminate endogenous protease activity. The host cells of the invention in some embodiments can produce N-glycans and/or O-glycans that do not comprise xylose in the glycan, or at least not in the core Man3 or Man4 structure.

In any of the embodiments the host cell or organism can contain a genetic modification to the alg3 gene and contain no genetic modification to any other gene encoding a mannosyl transferase. The glycomolecules produced by the host cells or organisms of the invention can be a glycoprotein, a glycopeptide, or a glycolipid.

In some embodiments the host cells or organisms contain a genetic modification in alg3, and except for alg3 can also contain all wild-type mannosyl transferase genes being expressed from the genome, and can contain no other expression of a mannosyl transferase gene, i.e. can also be free of any expression of mannosyl transferase from a plasmid or other nucleic acid construct.

### Oligosaccharyl Transferases (OSTs)

Oligosaccharyl transferases (OSTs) transfer sugar oligosaccharides to nascent proteins, or from a lipid-linked oligosaccharide (LLO) to the target protein or peptide. In some embodiments the oligosaccharide Glc3Man9GlcNAc2 is attached to an Asn residue in the sequence Asn-X-Ser or Asn-X-Thr where X is any amino acid except proline. OSTs can consist of one catalytically active subunit (Stt3) and several non-catalytic subunits that contribute to N-glycosylation by regulating substrate specificity, stability, or assembly of the complex. In some organisms several isoforms of the enzymes can exist and some organisms can lack some OST subunits. OSTs catalyze a reaction step in the N-linked glycosylation pathway. In various embodiments the OSTs useful in the invention can be protozoan OSTs, which can utilize a single-protein OST. Any of the OSTs can be overexpressed in an organism of the invention. Overexpression can mean that a gene is expressed in an increased quantity relative to normal expression, or expression at higher than wild type levels, or a level of expression that is higher than what is achieved without genetic manipulation. In one embodiment overexpression occurs by placing a sequence behind a strong promoter, which can be exogenous or endogenous. Endogenous OSTs, which can be overexpressed in the host cells or organisms of the invention include, but are not limited to, ChStt3 (SEQ ID NO: 34) from an organism of the family Thraustochytriaceae. Examples of protozoan OSTs include, but are not limited to, those from protozoa of the family Trypanosomatidae, These protozoa can be hemoflagellates and include the genera Crithidia, Herpetomonas, Leptomonas, Blastocrithidia, Phytomonas, Endotrypanum, Leishmania, and Trypanosoma sp. Species of these genera that are useful in the invention can be unicellular parasitic flagellate protozoa. Any expression of a heterologous gene is overexpression, since it is not normally expressed in the organism. The protozoan OSTs useful in the invention can be derived from species such as, for example, Leishmania brasiliensis, Leishmania major, or Trypanosoma brucei (e.g. Stt3A from T. brucei), Trypanosoma cruzi. Specific examples of protozoan OSTs useful in the invention include, but are not limited to, ThStt3A (SEQ ID NO: 28), ThStt3B (SEQ ID NO: 29), TbStt3C (SEQ ID NO: 30), LbStt3 1 (SEQ ID NO: 32), LbStt3 3 (SEQ ID NO: 33), LmStt3A, LmStt3B, LmStt3C, and LmStt3D (SEQ ID NO: 31). In some embodiments the OSTs can be those that are members of the Pfam family PF02516 and/or of the Pfam clan CL0111. In other embodiments the OSTs are members of the PPM superfamily 273, or of the Orientations of Proteins in Membranes (OPM) classified as members of the family 3rce, or are in the Carbohydrate-Active enzymes database (CAZy) classified as members of the family GT66. The OST can therefore be derived from a protozoa, meaning that it is found in the protozoa naturally, or that it comprises at least 90% sequence identity with an OST found naturally in a protozoa.

### Expression of OST

In any of the embodiments the host cells or organisms of the invention comprise and functionally express a nucleic acid sequence encoding the polypeptide sequence of a heterologous glycomolecule and functionally express a nucleic acid sequence encoding one or more heterologous oligosaccharyl transferase(s) (OSTs), or overexpress an endogenous OST. The heterologous glycomolecule can be expressed from an exogenous nucleic acid molecule, for example a plasmid or artificial chromosome, or in some embodiments can be integrated into and expressed from the host cell genome. The one or more OSTs can be provided on the same exogenous nucleic acid molecule as the sequence for the heterologous glycomolecule, or on a separate exogenous nucleic acid molecule. The sequence(s) encoding the OST(s) can also be inserted into the genome of the host cell. The sequence(s) encoding the OST(s) can also comprise a suitable promoter (and optionally a terminator) described herein for expressing the OST, or can be inserted in front of an endogenous promoter. The OST can be expressed from any of the sites described above, or from wherever it is provided. In some embodiments the OST can be genetically inserted (e.g. into the genome of the host cell) or can be the transformed into the host cell on one or more exogenous nucleic acid(s) (e.g. a plasmid) encoding one or more heterologous OST enzyme(s). The host cell can functionally express, produce, and optionally secrete, the encoded heterologous glycomolecule having a higher amount of glycan occupancy as described herein compared to the same glycomolecule produced by a corresponding host cell or organism not expressing the heterologous OST. The host cell can also functionally express and produce the heterologous OST (or overexpress an endogenous OST). In some embodiments the OST can be inserted behind a promoter on the genome of the host cell, and the promoter can be an endogenous promoter that regulates the heterologous OST. In one embodiment the promoter is inserted at a location on the genome where it will be expressed from an endogenous promoter. In one embodiment the OST gene can be inserted behind an endogenous actin promoter (SEQ ID NO: 41), although persons of ordinary skill with resort to this disclosure will realize many other promoters that will also function in the invention.

The host cells of the invention, in addition to expressing the OST sequence can also have the genetic modification to the one or more mannosyl transferase gene(s), as described herein. Thus, in one embodiment the host cells express a heterologous OST (or overexpress an endogenous OST), and also have a genetic modification to one or more mannosyl transferase gene(s) (e.g. alg3), and also express the heterologous glycomolecule. The genetic modification of a mannosyl transferase gene (e.g. a deletion or disruption of alg3) can also decrease the glycan occupancy on the heterologous glycomolecule produced. The glycan moieties on the heterologous glycomolecule can be N-glycan moieties or O-glycan moieties, or a combination of them both. Expression of the heterologous OST(s) results in the partial or complete restoration of glycan occupancy on the heterologous glycomolecule relative to the same heterologous glycomolecule produced by a corresponding cell not expressing the OST and cultivated under the same conditions. In various embodiments the glycan profile produced can have at least 25% or at least 30%, or at least 35%, or at least 40%, or at least 45%, or at least 50%, or at least 55%, or at least 60%, or at least 65%, or at least 75%, or at least 80%, or at least 90%, or at least 95% of the glycan sites on the heterologous glycomolecule occupied with a glycan, which can be an N-glycan and/or an O-glycan. The glycan sites can be those sites that are glycosylated when the glycomolecule is produced in a mammalian cell (e.g. a CHO cell). The glycans occupying the glycan sites can be Man3GlcNAc2, Man4GlcNAc2 and Man5GlcNAc2, or any combination or sub-combination of them, which are disclosed as if all possible combinations and sub-combinations are fully set forth herein. In various embodiments the cells with the genetic modification to the at least one mannosyl transferase gene and expressing the OST can also have 25% more, or at least 30% more, or at least 35% more, or at least 40% more, or at least 45% more, or at least 50% more, or at least 55% more, or at least 60% more, or at least 65% more, or at least 75% more, or at least 80% more, or at least 2× more, or at least 3× more, or at least 4× more occupancy of glycosylation sites relative to the same heterologous glycomolecule produced in a corresponding host cell that has the same genetic modification to the at least one mannosyl transferase gene and that does not express the OST.

A genetic modification can denote any one or more of a deletion, mutation, disruption, insertion, inactivation, attenuation, a rearrangement, an inversion, that results in a physical change to the modified gene or a regulatory sequence, and that reduces or eliminates expression of the one or more gene products. in various embodiments the genetic modification can be a deletion or a disruption. An unmodified nucleic acid sequence present naturally in the organism denotes a natural or wild type sequence. In any of the embodiments the genetic modification can be a deletion. As used herein a deletion can mean that at least part of the nucleic acid sequence is lost, but a deletion can also be accomplished by disrupting a gene through, for example, the insertion of another sequence (e.g. a selection marker), or a combination of deletion and insertion, but a deletion can also be performed by other genetic modifications. A deletion can mean that the gene no longer produces its functional gene product or, in various embodiments, that the gene produces less than 20% or less than 10% or less than 5% or less than 1% of its functional gene product versus production without the deletion under standard culturing conditions. The terms deletion cassette and disruption cassette are used interchangeably.

In some embodiments N-glycans occupying the glycan sites can include, but are not limited to, Man3GlcNAc2, and/or Man4GlcNAc2, and/or Man5GlcNAc2, or any combination or sub-combination of them, which are disclosed as if set forth fully herein in all possible combinations. These glycans can be present on a glycomolecule as disclosed herein. These glycans can be present on a glycomolecule as disclosed herein.

### Heterologous Glycomolecules

Glycoproteins and glycopeptides have one or more carbohydrate groups attached to their polypeptide chain. In some embodiments the heterologous glycomolecule produced by the cells or organisms of the invention can be a therapeutic molecule, such as a glycoprotein, glycopeptide, or glycolipid therapeutic molecule, e.g. enzymes, Ig-Fc-Fusion proteins, or an antibody. The antibody can be a functional antibody or a functional fragment of an antibody. In various embodiments the antibody can be alemtuzumab, denosumab, eculizumab, natalizumab, cetuximab, omalizumab, ustekinumab, panitumumab, trastuzumab, belimumab, palivizumab, natalizumab, abciximab, basiliximab, daelizumab, adalimumab (anti-TNF-alpha antibody), tositumomab-Il31, muromonab-CD3, canakinumab, infliximab, daclizumab, tocilizumab, thymocyte globulin, anti-thymocyte globulin, or a functional fragment of any of them. The glycoprotein can also be alefacept, rilonacept, etanercept, belatacept, abatacept, follitropin-beta, or a functional fragment of any of them. The antibody can also be any antiTNF-alpha antibody or an anti-HER2 antibody, or a functional fragment of any of them. The glycoprotein can be an enzyme, for example idursulfase, alteplase, laronidase, imiglucerase, agalsidase-beta, hyaluronidase, alglucosidase-alfa, GalNAc 4-sulfatase, pancrelipase, or DNase. The protein can be an antibody and/or a therapeutic protein, and can also be a monoclonal antibody. A functional antibody (or immunoglobulin) or fragment of an antibody binds to a target epitope and thereby produces a response, for example a biological response or action, or the cessation of a response or action. The response can be the same as the response to a natural antibody, but the response can also be to mimic or disrupt the natural biological effects associated with ligand-receptor interactions. When the heterologous glycomolecule is an immunoglobulin or antibody the glycan sites can be comprised on the heavy chain of the immunoglobulin.

When the protein is a functional fragment of an antibody it can comprise at least a portion of the variable region of the heavy chain, or can comprise the entire antigen recognition unit of an antibody, but nevertheless comprise a sufficient portion of the complete antibody to perform the antigen binding properties that are similar to or the same in nature and affinity to those of the complete antibodies. In various embodiments a functional fragment of a glycoprotein, glycopeptide, glycolipid, antibody, or immunoglobulin can comprise at least 10% or at least 20% or at least 30% or at least 50% or at least 60% or at least 70% or at least 80% or at least 90% or at least 95% of the native sequence, and optionally any functional fragment can also have at least 70% or at least 80% or at least 90% or at least 95% sequence identity to that indicated portion of the native sequence; for example, a functional fragment can comprise at least 85% of the native antibody sequence, and have a sequence identity of at least 90% to that 85% portion of the native antibody sequence. Any of the recombinant cells disclosed herein can comprise a nucleic acid encoding a functional and/or assembled antibody molecule described herein, or a functional fragment thereof.

In various embodiments the glycomolecule can be a hormone, e.g., human growth hormone, leutinizing hormone, thyrotropin-alpha, interferon, darbepoetin, erythropoietin, epoetin-alpha, epoetin-beta, FS factor VIII, Factor VIIa, Factor IX, anithrombin/ATIIcytokines, clotting factors, insulin, erythropoietin (EPO), glucagon, glucose-dependent insulinotropic peptide (GIP), cholecystokinin B, enkephalins, and glucagon-like peptide (GLP-2) PYY, leptin, and antimicrobial peptides. In any of the embodiments the glycomolecule can be encoded on DNA exogenous to the cell, e.g. a plasmid, artificial chromosome, other extranuclear DNA, or another type of vector DNA. It can also be present on an exogenous sequence inserted into the cellular genome.

As used herein, the terms “percent identity” or “homology” with respect to nucleic acid or polypeptide sequences are defined as the percentage of nucleotide or amino acid residues in the candidate sequence that are identical with the known polypeptides, after aligning the sequences for maximum percent identity and introducing gaps, if necessary, to achieve the maximum percent homology. N-terminal or C-terminal insertion or deletions shall not be construed as affecting homology, and internal deletions and/or insertions into the polypeptide sequence of less than about 30, less than about 20, or less than about 10 amino acid residues shall not be construed as affecting homology. Homology or identity at the nucleotide or amino acid sequence level can be determined by BLAST (Basic Local Alignment Search Tool) analysis using the algorithm employed by the programs blastp, blastn, blastx, tblastn, and tblastx (Altschul (1997), Nucleic Acids Res. 25, 3389-3402, and Karlin (1990), Proc. Natl. Acad. Sci. USA 87, 2264-2268), which are tailored for sequence similarity searching. The approach used by the BLAST program is to first consider similar segments, with and without gaps, between a query sequence and a database sequence, then to evaluate the statistical significance of all matches that are identified, and finally to summarize only those matches which satisfy a preselected threshold of significance. For a discussion of basic issues in similarity searching of sequence databases, see Altschul (1994), Nature Genetics 6, 119-129. The search parameters for histogram, descriptions, alignments, expect (i.e., the statistical significance threshold for reporting matches against database sequences), cutoff, matrix, and filter (low complexity) can be at the default settings. The default scoring matrix used by blastp, blastx, tblastn, and tblastx is the BLOSUM62 matrix (Henikoff (1992), Proc. Natl. Acad. Sci. USA 89, 10915-10919), recommended for query sequences over 85 in length (nucleotide bases or amino acids).

For blastn, designed for comparing nucleotide sequences, the scoring matrix is set by the ratios of M (i.e., the reward score for a pair of matching residues) to N (i.e., the penalty score for mismatching residues), wherein the default values for M and N can be +5 and −4, respectively. Four blastn parameters can be adjusted as follows: Q 10 (gap creation penalty); R 10 (gap extension penalty); wink 1 (generates word hits at every winkth position along the query); and gapw 16 (sets the window width within which gapped alignments are generated). The equivalent Blastp parameter settings for comparison of amino acid sequences can be: Q 9; R 2; wink 1; and gapw 32. A BESTFIT● comparison between sequences, available in the GCG package version 10.0, can use DNA parameters GAP 50 (gap creation penalty) and LEN 3 (gap extension penalty), and the equivalent settings in protein comparisons can be GAP 8 and LEN 2.

When referring to the nucleic acid or polypeptide sequences of the heterologous glycomolecules or OSTs in the present disclosure, included in the disclosure are sequences considered to be derived from the original sequence. Sequences disclosed therefore include nucleic acid and polypeptide sequences having sequence identities of at least 40%, at least 45%, at least 50%, at least 55%, of at least 60%, at least 65%, at least 70%, at least 75%, at least 80%, or at least 85%, for example at least 86%, at least 87%, at least 88%, at least 89%, at least 90%, at least 91%, at least 92%, at least 93%, at least 94%, at least 95%, at least 96%, at least 97%, at least 98%, at least 99%, or about 100% or 85-99% or 85-95% or 90-99% or 95-99% or 97-99% or 98-99% sequence identity with the full-length polypeptide or nucleic acid sequence of any of SEQ ID Nos: 1-41, and fragments thereof. Fragments of sequences can include sequences having a consecutive sequence of at least 20, or at least 30, at least 50, at least 75, at least 100, at least 125, 150 or more, or 20-40 or 20-50 or 30-50 or 30-75 or 30-100 amino acid residues of the entire protein, or at least 100 or at least 200 or at least 300 or at least 400 or at least 500 or at least 600 or at least 700 or at least 800 or at least 900 or at least 1000 or 100-200 or 100-500 or 100-1000 or 500-1000 or any of these amounts but less than 500 or less than 1000 or less than 2000 consecutive nucleotides of any of SEQ ID Nos. 1-41. Also disclosed are variants of such sequences, e.g., wherein at least one amino acid residue has been inserted N- and/or C-terminal to, and/or within, the disclosed sequence(s) which contain(s) the insertion and substitution. Contemplated variants can additionally or alternately include those containing predetermined mutations by, e.g., homologous recombination or site-directed or PCR mutagenesis, and the corresponding polypeptides or nucleic acids of other species, including, but not limited to, those described herein, the alleles or other naturally occurring variants of the family of polypeptides or nucleic acids which contain an insertion and substitution; and/or derivatives wherein the polypeptide has been covalently modified by substitution, chemical, enzymatic, or other appropriate means with a moiety other than a naturally occurring amino acid which contains the insertion and substitution (for example, a detectable moiety such as an enzyme).

### Promoters and Terminators

Promoters and terminators can be used on expression cassettes or other nucleic acid constructs in the invention or inserted behind a gene to be overexpressed, and the promoter (and terminator) can be any suitable promoter and/or terminator. Promoters and/or terminators disclosed herein can be used in any combination or sub-combination. For example, any promoter described herein (or other promoters that may be isolated from or functional in the host cell or organism), or derived from such sequences, can be used in combination with any terminator described herein or other terminators functional in the recombinant cell or organism, or derived from such sequences. For example, promoter and terminator sequences may be derived from organisms including, but not limited to, Heterokonts (including Labyrinthulomycetes), organisms of the family Thraustochytriaceae, yeast or other fungi, microalgae, algae, and other eukaryotic organisms. In various embodiments the promoter and/or terminator is any one operable in a cell or organism that is a Labyrinthulomycetes cell, including any Family (e.g. Thraustochytriaceae) or a genus thereof. Any of the constructs can also contain one or more selection markers, as appropriate. A large number of promoters and terminators can be used with the host cells of the invention. Those described herein are examples and the person of ordinary skill with resort to this disclosure will realize or be able to identify other promoters useful in the invention. Examples of promoters that can be utilized in the invention include the alpha-tubulin promoter, actin promoter, TEF, TEF1, hsp60, hsp60-788 promoter, hsp70, RPL11, Tsp-749 promoter, Tubu738 promoter, Tubu-997 promoter, a promoter from the polyketide synthase system, and a fatty acid desaturase promoter. Examples of useful terminators include pgkl, CYCl, and eno2. Promoters and terminators can be used in any advantageous combination and all possible combinations of these promoters and terminators are disclosed as if set forth fully herein.

In some embodiments the expression cassettes utilized in the invention comprise any one or more of 1) one or more signal sequences; 2) one or more promoters; 3) one or more terminators; and 4) an exogenous sequence encoding one or more proteins, which can be a heterologous protein; 4) optionally, one or more selectable markers for screening on a medium or a series of media or other growth conditions. These components of an expression cassette can be present in any combination, and each possible sub-combination is disclosed as if fully set forth herein. In specific embodiments the signal sequences can be any described herein, but can also be other signal sequences. Various signal sequences for a variety of host cells are known in the art, and others can be identified with reference to the present disclosure and which are also functional in the host cells being utilized. In exemplary specific embodiments the promoter can be an alpha-tubulin promoter or TEFp. Any promoter disclosed herein can be paired with any suitable terminator, but in specific embodiments the tub-alpha-p can be paired with the pgk1 terminator. In another embodiment the TEFp promoter can be paired with the eno2 terminator, both terminators being from Saccharomyces cerevisiae and also being functional in Labyrinthulomycetes. The selectable marker can be any suitable selectable marker or markers but in specific embodiments it can be nptII or hph. In one embodiment nptII can be linked to the heavy chain constructs and hph can be linked to the light chain constructs.

The present invention also provides a nucleic acid construct, which can be an insertion cassette for performing an insertion of an OST gene and/or another heterologous gene described herein. The nucleic acid construct can have a sequence encoding an OST gene described herein functional in a Labyrinthulomycetes host cell, a promoter, and optionally, a terminator, both functional in the host cell. The nucleic acid construct can also be a mutation or modification cassette for performing a mutation, or other genetic modification in a gene, which can be any gene described herein (homologous or heterologous). The nucleic acid construct can be regulated by the promoter sequence and, optionally, the terminator sequence functional in a host cell. The host cell can comprise an expression cassette and also an insertion, mutation, or other modification cassette as disclosed herein, and can also be a CRISPR/Cas 9 cassette that can modify any one or more of the target genes as disclosed herein. The construct or cassette can also have a sequence encoding 5′ and 3′ homology arms to the gene, which in some embodiments can be an OST. The construct can also have a selection marker, which in one embodiment can be nat but any appropriate selection marker can be used.

In any of the embodiments OSTs or other genes can be overexpressed. Overexpression of genes can be achieved by adding additional copies of the gene, such as two or more, or three or more, or four or more, or five or more copies of the gene. For endogenous genes more copies can be added to the genome, or for any of the genes overexpression can involve expressing the gene from a plasmid or other nucleic acid construct, which can be inserted into the genome. Overexpression can also involve expressing genes (endogenous or heterologous) from a stronger promoter than the native (endogenous) promoter. In one embodiment any of the OST genes can be overexpressed utilizing the actin promoter (SEQ ID NO: 41).

### Additional Modifications

In any of the embodiments disclosed herein the recombinant cells or organisms of the invention can express an OST gene as disclosed herein, and can also contain a genetic modification to one or more gene(s) that encode a mannosyl transferase enzyme. As a result of the modification the cells produce a heterologous glycomolecule that has an N-linked glycan profile that is more simplified, e.g. having a Man3 and/or Man4 structure. In some embodiments the glycomolecule has at least 25% or at least 35%, or at least 40%, or at least 45% or at least 50%, or at least 55%, or at least 60%, or at least 65%, or at least 70%, or at least 75%, or at least 80%, or at least 85%, or at least 90% fewer high mannose (Man5 core and higher) N-glycan structures compared to the same molecule produced by a corresponding cell that does not have the modification to the one or more mannosyl transferase gene(s) and cultivated under the same conditions, i.e. a reference cell.

As a result of the genetic modification to the one or more gene(s) that encode a mannosyl transferase enzyme (e.g. the alg3 gene), the cells can also produce a heterologous glycoprotein or glycopeptide that has lower glycan occupancy. However, the expression of the one or more heterologous OSTs can at least partially or fully restore the glycan occupancy to the heterologous glycoprotein or glycopeptide (e.g. an immunoglobulin).

The host cells or organisms of the invention having the genetic modification to the one or more mannosyl transferase gene(s) provide the important benefit that at least 10% or at least 20% or at least 30%, or at least 40%, or at least 50% or at least 60% or at least 70%, or at least 75%, or at least 80%, or at least 85%, or at least 90% of the N-glycans are Man3 in one embodiment, are Man4 in another embodiment, or a combination of Man3 and Man4 structures in another embodiment. The cells or organisms can also have at least 20% more, or at least 30% more, or at least 40% more, or at least 50% more, or at least 60% more, or at least 70% more or at least 80% more, or at least 90% more, or at least 2×, or at least 4×, or at least 5×, or at least 7× more, or at least 8× more, or at least 9× more, or at least 10× more, or at least 11× more, or at least 12× more, or at least 13× more, or at least 15× more, or at least 17× more, or at least 20× more, or at least 22× more, or at least 25× more, or at least 27× more, or at least 28× more Man3 in one embodiment, Man4 in another embodiment, or a combination of Man3 and Man4 (paucimannose) in another embodiment, compared to a reference cell not having the genetic modification to at least one mannosyl transferase gene and cultivated under the same conditions.

In any of the embodiments the genetic modification can be to any one or more of the alg3 gene(s) or to any one or more gene(s) in the mannosyl transferase gene family, or in a regulatory sequence affecting expression of the gene (e.g. in a promoter), but can also be in a non-regulatory sequence. Members of this family include, but are not limited to, alg1, alg2, alg3, alg6, alg8, alg9, alg10, alg 11, alg13, and alg14. In one embodiment the genetic modification is a deletion or disruption but can be any genetic modification, which can be present in any one or more genes of the mannosyl transferase gene family, or in any combination or sub-combination of them, which is disclosed as if set forth fully herein in all possible combination and sub-combinations. The host cell can be a cell of the invention described herein. Therefore, the proteins produced avoid many of the problems associated with the use of glycoproteins, glycopeptides, or glycolipids having patterns of glycosylation of non-human species. When combined with the expression of one or more genes encoding an OST in the host cell as described herein, further benefit is realized by further humanizing the glycomolecule by reducing or removing sulfate moieties on the N-glycan structures.

The mannosyl transferase genes that can be modified in the invention can include any one or more of an alpha-1,2-mannosyl transferase, an alpha-1,3-mannosyltransferase, or an alpha-1,6-mannosyltransferase. Any one or more of these genes can be present as more than one copy and the cells and methods can have the genetic modification to all copies of the gene.

In one embodiment the deletion, disruption, or other genetic modification is to one or more alg3 gene(s), which encodes an enzyme that catalyzes the addition of the first dol-P-Man derived mannose in an alpha-1,3 linkage to Man5GlcNAc2-PP-Dol. Genes that are members of the Alg3 sub-family encode an alpha-1,3-mannosyl transferase and are found in fungi, mammals, yeast, Labyrinthulomycetes (e.g. Thraustochytriaceae (including but not limited to Schizochytriwn, Aurantiochytrium, Thraustochytrium) and other Labyrinthulomycetes), and a wide variety of other organisms. In a specific embodiment the modification is a deletion or knock out or disruption of one or more alg3 gene(s), which can be done in a host cell of the Thraustochytriaceae family, such as a Schizochytrium or Aurantiochytrium. Some cells contain more than one Alg3 gene and the deletion, knock out, or disruption can be in any one or more of the Alg3 genes, or all of the Alg3 genes.

Additional modifications and information can be found in U.S. application Ser. No. 15/799,785, filed Oct. 31, 2017; and U.S. application Ser. No. 15/967,202, filed Apr. 30, 2018, both of which are hereby incorporated by reference in their entireties, including all tables, figures, and claims.

### Methods

The invention also provides methods of producing glycomolecules in host cells described herein that have a glycan profile having high N-glycan and/or O-glycan occupancy, as described herein. The methods can involve any one or more steps of: transforming a host cell with a vector (e.g. an expression vector) or other exogenous nucleic acid encoding a heterologous glycomolecule for expression from the vector or from a site integrated into the chromosome of the cell; optionally, a step of transforming the host cell with a vector (e.g, an expression vector) or other exogenous nucleic acid encoding a heterologous OST for expression from the vector or for integration into and expression from the genome of the cell, or optionally a step of performing a genetic modification to one or more native OST gene(s) to overexpress the gene(s); or a step of inserting a promoter described herein behind a sequence encoding an endogenous OST; cultivating the cell; and harvesting the heterologous glycomolecule that has a glycan profile with higher N-glycan and/or O-glycan occupancy as described herein. Optionally the method can also have a step of performing a deletion, disruption, or other genetic modification to one or more mannosyl transferase genes as described herein. Instead of performing these steps one can perform a step of obtaining a host cell having the stated characteristics, as described above.

Any of the methods can optionally include a step of deleting or disrupting in the host cell one or more mannosyl transferase genes described herein, which can be the alg3 gene. The heterologous OST gene can be a protozoan OST. The glycomolecule can be an immunoglobulin, an antibody, or any heterologous glycomolecule described herein.

In one embodiment any of the methods can also involve transforming a host cell with an expression cassette, mutation cassette, or modification cassette to thereby transform the host cell with a heterologous OST (or mutate a native OST) as disclosed herein, expressing the heterologous OST, performing a genetic modification to a mannosyl transferase gene in the host cell (e.g. a deletion or disruption), cultivating the cell, and harvesting a glycomolecule that has a glycan profile with higher N-glycan and/or O-glycan occupancy as described herein.

### Compositions

The present invention also provides compositions comprising a glycomolecule produced by and derived from a recombinant cell or organism described herein, wherein the glycomolecule has a glycan profile with at least 10% or at least 20% or at least 30%, or at least 40%, or at least 50% or at least 60% or at least 70%, or at least 75%, or at least 80%, or at least 85%, or at least 90% of the N-glycans being Man3, or Man4, or a combination of Man3 and Man4 structures, The compositions also contain a heterologous glycomolecule described herein having at least 30%, or at least 40%, or at least 50% or at least 60% or at least 70%, or at least 75%, or at least 80%, or at least 85%, or at least 90% glycan occupancy.

The glycan profile can be an N-glycan profile, an 0-glycan profile, or both. The composition can be produced by and derived from a recombinant Labyrinthulomycete cell or any organism described herein. Derived from a cell means that the glycomolecule was synthesized by the cell, and optionally harvested. In some embodiments the entire glycomolecule was synthesized by the cell, including the glycan portion. The cell that produces the glycomolecule can comprise and express a heterologous OST and, optionally, a genetic modification in one or more genes that encode mannosyl transferase genes, as described herein. The composition can be any of the compositions derived from host cells, as described herein.

The present invention also provides compositions containing a therapeutic glycomolecule produced by the cells or organisms of the invention described herein. A therapeutic glycomolecule can be one useful for a therapeutic purpose in a human or animal patient. The therapeutic glycomolecule contained in the composition can be any described herein, for example an antibody, an immunoglobulin, a single domain antibody, or any therapeutic protein described herein. Non-limiting examples include natalizumab and trastuzumab (SEQ ID Nos: 3-4). The therapeutic glycomolecule can be provided in a pharmaceutically acceptable carrier.

### Organisms

Persons of ordinary skill in the art are able to isolate Labyrinthulomycetes organisms described herein in various coastal marine habitats, such a salt marshes and mangrove swamps (e.g. found in tropical regions). For the present invention cells of the taxonomic family Thraustochytriaceae (Aurantiochytrium sp.) were isolated from a sample obtained from a mangrove lagoon in a tropical area of Mexico using a plankton tow (10 um). Organisms harvested were cultured on a media containing sea water, glucose, yeast extract and peptone, and standard enrichment steps were carried out on the same media. A single colony isolate was selected that was found to be amenable to producing and secreting proteins and was used as the base strain (designated #6267).

Table of Strains

#6267-base Aurantiochytrium sp. Strain

#5942 trastuzumab-producing strain

#5950/1 trastuzumab-producing strains

#6456 trastuzumab-producing and carrying Cas9

#6669/70-#6456 with alg3 deletion

### Glycan Determination

Glycan occupancy for immunoglobulins can be determined by first purifying the final product over a protein A column. The purified samples were digested with IdeS protease to generate a specific and reproducible fragment of the IgG Fc portion. The samples were chromatographed on C8 resin via HPLC. Masses were recorded via an in-line Agilent● 6520 QTof mass spectrometer and deconvolution was conducted using the manufacturer's software. The intact mass of the aglycosylated Fc fragment was calculated based on its amino acid sequence and the calculated mass was used to identify it. Specific glycoforms of known mass were identified by calculating the difference between the observed mass of the glycosylated fragment and that of the aglycosylated fragment. All glycosylated and aglycosylated fragments were quantified based on their respective deconvoluted peak heights and each individual form was expressed as a percent of the total of all forms detected. Occupancy was calculated as 100% minus the percent of the IdeS Fc fragment that was aglycosylated.

### EXAMPLES

### Example 1

### Construction of alg3 Deletion Strain Expressing Trastuzumab (#6670)

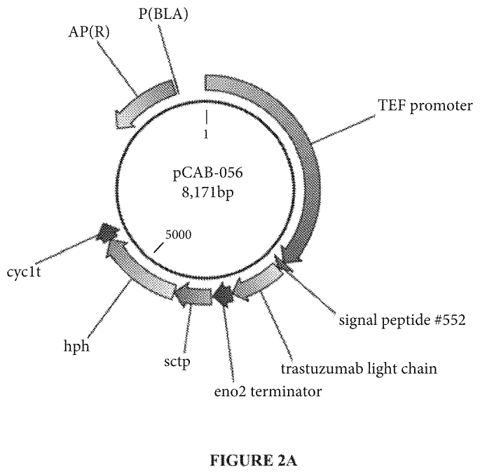

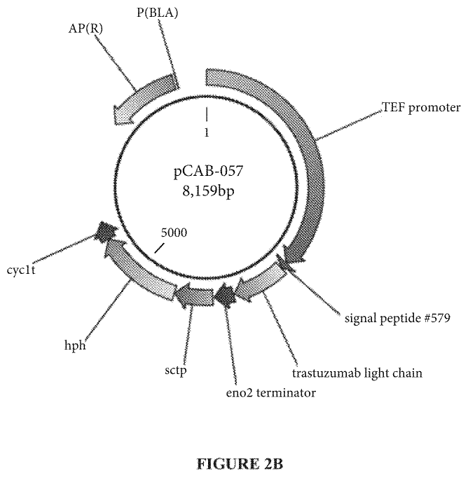

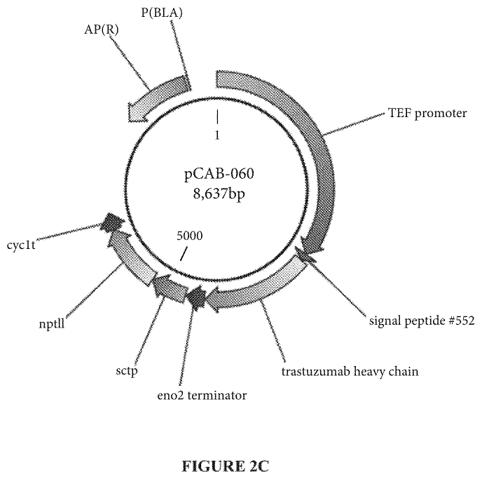

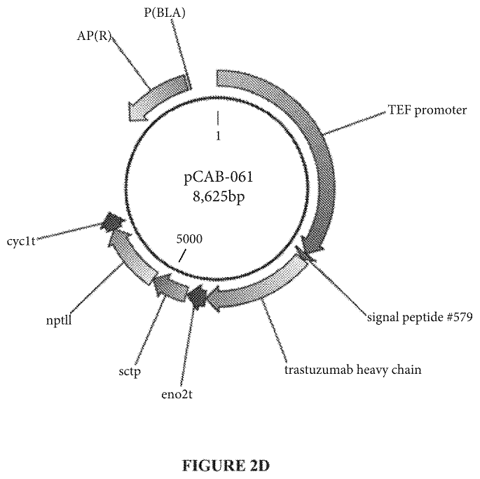

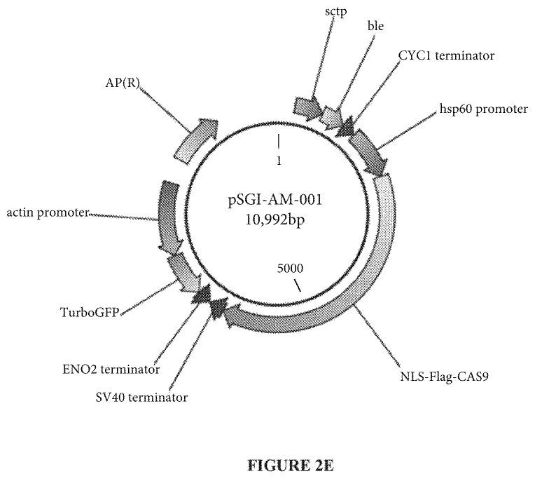

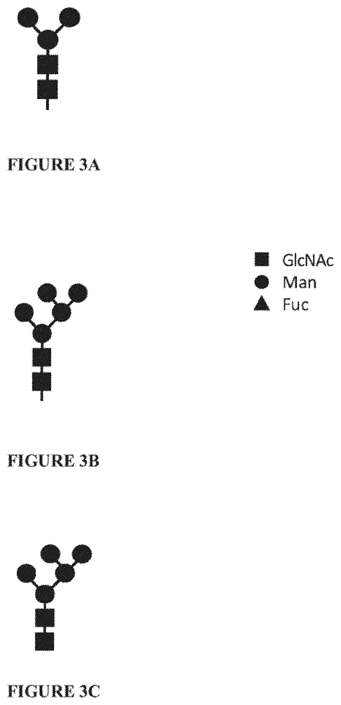

Constructs pCAB056 (FIG. 2a) is a chytrid expression cassette for the TEF promoter (SEQ ID NO: 1) driven expression of the trastuzumab light chain (SEQ ID NO: 3) where secretion is mediated by signal peptide #552 (SEQ ID NO: 25). This cassette carries the hph marker for selection in Thraustochytriaceae organisms.

Constructs pCAB057 (FIG. 2b) is a chytrid expression cassette for the TEF promoter driven expression of the trastuzumab light chain where secretion is mediated by signal peptide #579 (SEQ ID NO: 2). This cassette carries the hph marker for selection in Thraustochytriaceae organisms.

Constructs pCAB060 (FIG. 2c) is a chytrid expression cassette for the TEF promoter driven expression of the trastuzumab heavy chain (SEQ ID NO: 4) where secretion is mediated by signal peptide #552 (SEQ ID NO: 25). This cassette carries the nptII marker for selection in Thraustochytriaceae organisms.

Constructs pCAB061 (FIG. 2d) is a chytrid expression cassette for the TEF promoter driven expression of the trastuzumab heavy chain where secretion is mediated by signal peptide #579 (SEQ ID NO: 2). This cassette carries the nptII marker for selection in Thraustochytriaceae organisms.

Chytrid strains expressing trastuzumab was produced by co-transforming Aurantiochytrium sp. #6267 with pCAB056, 057, 060 and 061 that had been linearized by AhdI digestion, Transformants that were resistant to both Hygromycin B and Paromomycin were screened by ELISA for production of antibody. Each clone was cultured overnight in 3 ml FM2 (17 g/L Instant Ocean™, 10 g/L yeast extract, 10 g/L peptone, 20 g/L dextrose) in a 24-well plate. They were then diluted 1000× into fresh FM2 (3 mL) and incubated for about 24 hours. The cells were pelleted by centrifugation (2000 g×5 min) and the supernatants assayed for the presence of antibody by HC-capture/LC-detect sandwich ELISA. The transformants were also screened for the signal peptide that had been introduced into the strain by colony PCR.

Trastuzumab titers in the top three producing strains were measured by ELISA. The results are shown in the Table below. The signal peptide present in these strains are also shown with the strain ID numbers.

TABLE 1
trastuzumab titers

| | | Signal | Signal | |
|---|---|---|---|---|
| | strain | peptide | peptide | Titers |
| Clone # | ID # | on LC | on HC | (mg/L) |
| Her.1.2 | 5942 | 579 | 579 | 30 |
| Her.2.24 | 5950 | 579 | 552, 579 | 16 |
| Her.2.40 | 5951 | 579 | 579 | 31 |

### Example 2

### Cas9 Expression Constructs

Constructs pSGI-AM-001 (SEQ ID NO: 5) is an expression cassette for Cas9. This cassette carries sequences for the constitutive expression of Cas9 from Streptococcus pyogenes under the control of the hsp60 promoter (SEQ ID NO: 6). This construct also carries the TurboGFP reporter and the ble marker (FIG. 2e).

### Example 3

### Construction of Trastuzumab-Producing Strain Carrying Cas9 (#6456)

CAS9 was introduced into the trastuzumab producing strain #5942 by transforming this strain with the cassette pAM-001 linearized by digestion by AhdI. Zeocin™ resistant clones were examined for production of trastuzumab by ELISA. Each clone was cultured overnight in 3 mL FM2 (17 g/L Instant Ocean™, 10 g/L Yeast extract, 10 g/L Peptone, 20 g/L dextrose) in a 24-well plate. 10 μL of this culture was used to inoculate fresh FM2 (3 mL) and incubated for about 24 hours. The cells were pelleted by centrifugation (2000 g×5 min) and the supernatants assayed for the presence of antibody by HC-capture/LC-detect sandwich ELISA. Transformants producing trastuzumab were also screened for the presence of the CAS9 expression cassette by PCR using primers oSGI-JU-1360 (SEQ ID NO: 7) and oSGI-JU-0459 SEQ ID NO: 26). One of these clones that produced trastuzumab at similar levels as the parent strain #5942 and was positive for the CAS9 expression cassette was designated #6456.

### Example 4

### Construction of alg3 Deletion Cassettes

The disruption cassette utilized to delete or disrupt alg3 was a linear fragment of DNA having three parts, from 5′ to 3′: 1) a 5′ homology arm, 2) a selection marker and 3) a 3′ homology arm. The 5′ homology arm can be a region of 500 1000 bp found upstream in the genome of the sequence being targeted for deletion. Selection markers generally contain a sequence encoding for expression of a gene (i.e. an antibiotic resistance gene) that allows for selection of successful transformants. The 3′ homology arm can be a region of 500 1000 bp found downstream in the genome of the sequence being targeted for deletion.

This example describes the construction of a disruption cassette of the alg3 gene in Aurantiochytrium sp. Three translation IDs (SG4EUKT579099, SG4EUKT579102, and SG4EUKT561246) (SEQ ID Nos: 11-13, respectively) were found in the genome assembly of the Aurantiochytrium sp. base strain (#6267). All three sequences encode a 434 amino acid protein (mannosyl transferase) (SEQ ID Nos: 8-10). SG4EUKT579099 and SG4EUKT579102 are identical at both the amino acid and nucleotide levels. SG4EUKT561246 shares greater than 99% identity to the other sequences at both the amino acid and nucleotide levels. This high level of identity allowed for the targeting of all three sequences using Cas9 and a single guide RNA (gRNA) sequence (SEQ ID NO: 14) as well as a single disruption cassette (alg3::nat) comprised of a selectable marker (nat) providing resistance to nourseothricin that is flanked by 5′ and 3′ alg3 homology arms (500-about 1000 bp).

The alg3::nat disruption cassette was generated by amplifying the 5′ and 3′ alg3 homology arms from the base strain (#6267) genomic DNA, while the selectable marker (nat) was amplified from a plasmid carrying a Thraustochytriaceae expression cassette for nat. The nat marker was amplified using primers oSGI-JU-0017 (SEQ ID NO: 17) and oSGI-JU-0001 (SEQ ID NO: 18). The 5′ homology arm was amplified using primers oCAB-0294 (SEQ ID NO: 19) and oCAB-0295 (SEQ ID NO: 20), the latter has a 5′ extension that is complementary to oSGI-JU-017. The 3′ homology arm was amplified using primers oCAB-0296 (SEQ ID NO: 21) and oCAB-0297 (SEQ ID NO: 22), the former has a 5′ extension that is complementary to oSGI-JU-0001. The three fragments were assembled, also by PCR using primers oCAB-0294 and pCAB-0297. The purified PCR product was used for transformations.

gRNA was generated using the commercially available MEGAshortscript™ T7 kit, but an RNAse inhibitor was added to the reaction mix. Template was generated by annealing together oligonucleotides oCAB-0341 and oCAB-0342 (SEQ ID Nos: 15-16, respectively).

### Example 5

### Deletion of alg3

Genome editing for a deletion of a gene can be carried out by transforming the host strain expressing Cas9 with a gRNA targeting a specific site in the genome and a disruption cassette generated using homology arms flanking this site. Homology arms are designed to delete several hundred bases from the genomic sequence.

Deletion of alg3 in the trastuzumab Cas9 clone #6456 was carried out by transforming this strain with a linear alg3::nat disruption cassette and gRNA. Nourseothricin resistant colonies were screened for the deletion of alg3 by quantitative PCR (qPCR) using primers oCAB-0298 & oCAB-0299 (SEQ ID Nos: 23-24, respectively). Four clones were identified that had alg3 deleted and were designated strain IDs #6667-#6670.

### Example 6

### Antibody Production

Strain #6456 and the four alg3 deletion clones described above were cultivated in 24 well plates for 22 hours and the trastuzumab levels in the supernatant were determined by ELISA. IgG-ELISA was determined by coating the plates with unlabeled mouse anti-human-IgG capture antibody followed by incubation with detection antibody mouse anti-human kappa-HRP. Deletion of alg3 did not have a negative effect on antibody titers.

TABLE 2
trastuzumab titers in cultures
of alg3 deleted clones

| | trastuzumab |
|---|---|
| Strain | Titers |
| ID | (mg/L) |
| #6456 | 6.6 |
| #6667 | 6.9 |
| #6668 | 7.5 |
| #6669 | 7.0 |
| #6670 | 9.8 |

### Example 7

### OST Overexpression in Labyrinthulomycetes Cells

OST (Stt3) genes were identified and codon optimized (Table 3) for overexpression in wild type Labyrinthulomycetes strain #6267. Sequences were obtained from databases such as the Archetype● database or the UniProt● database.

TABLE 3
OST genes and identifying references from databases

| OST | | |
|---|---|---|
| gene | source | |
| name | organism | Reference |
| ChStt3 | wild type | SG4EUKT566306 |
| | (#6267) | (Archetype● #) |
| LbStt3_3 | Leishinania | A4HMD7 |
| | brasiliensis | (UniProt●) |

OST genes were codon optimized for expression in Labyrinthulomycetes (Aurantiochytrium sp.) cells using the commercially available Archtype™ optimization tool and cloned behind the actin promoter and in front of the ENO2 terminator. The constructs also carried the bsr marker. The constructs were linearized by restriction digestion within the actin promoter sequence and transformed into #6670. By cutting within the promoter sequence, the integration of the expression cassette was targeted to the endogenous actin promoter sequence. The resulting transformants were screened for integration at the actin promoter sequence by colony PCR for 5′ and 3′ junctions between the cassette and the external genomic sequence. Production of trastuzumab was confirmed by ELISA analysis.

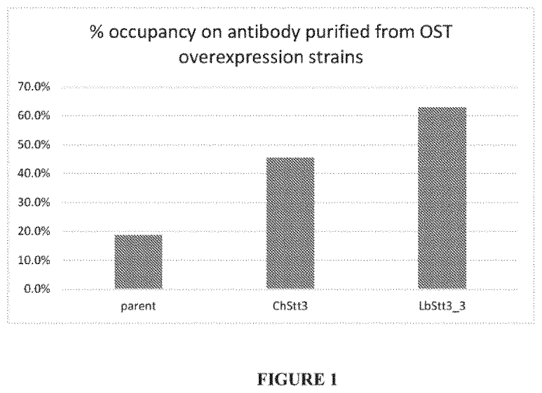

The cells also expressed and produced the heterologous glycoprotein antibody trastuzumab. The strains overexpressing the Labyrinthulomycetes wild type ChStt3 and the LbStt3 3 OSTs were used to produce trastuzumab in a shake flask fermentation. The final product was purified over a protein A-column and its glycosylation determined by intact mass analysis. The purified samples were digested with trypsin and analyzed using 1-dimensional nano LC-MS/MS. Occupancy was calculated as 100% minus the percent that was aglycosylated. Data is graphically depicted in FIG. 1. The bars show the percentage of glycosylation sites on trastuzumab occupied by an N-glycan. The parent cell (not expressing OST) had a glycan occupancy level of 18.7%; the cell overexpressing ChStt3 had a glycan occupancy level of 45.5%; and the cell expressing the heterologous LbStt3 3 had a glycan occupancy level of 62.9%. The data therefore illustrate that expression of a heterologous OST (or overexpression of an endogenous OST) results in a significant increase in glycan occupancy on the antibody produced by the cell.

### Example 8

### Glycan Analysis

Purified antibodies produced by the Alg3+ and Alg3− strains were analyzed by release of glycans using PNGaseF and PNGaseA and analysis by MALDI TOF/TOF and ESI-MS. The analysis of all data give a complete picture of the number and abundance of all glycans present in each sample, as well as the structures in each sample.

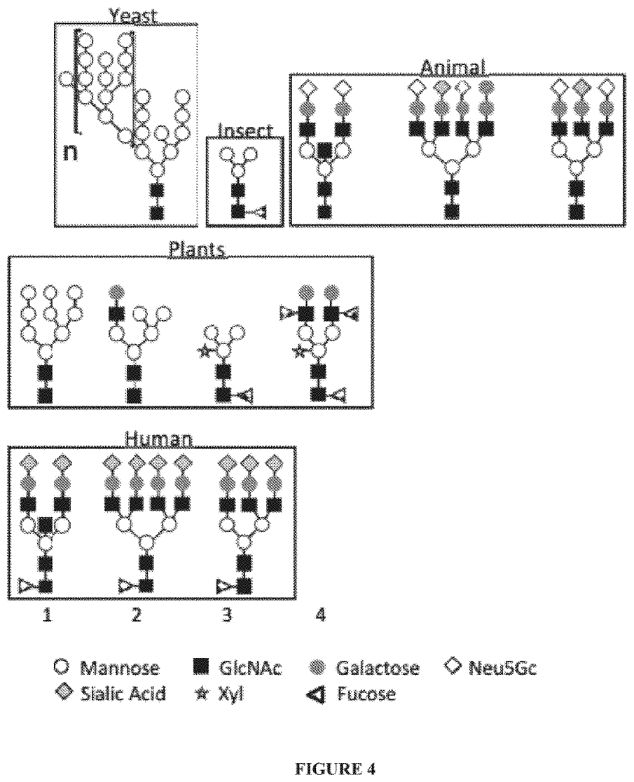

The combined data from the previous analyses confirmed that N-linked glycosylation in both samples only occurred at the expected site, Asn327. There was no detectable O-glycosylation in either sample, A large number of high mannose glycans, some of which contained xylose and sulfated structures, were detected on antibody from Alg3+ strain; whereas far fewer N-linked glycans were observed on sample from Alg3− strain (FIGS. 4-5, Tables 6 and 7). None of the N-linked glycans produced by Alg3− contain xylose, fucose, or galactose. The majority of the N-linked glycans produced by Alg3− have a Man3 structure.

The combined data from the previous analyses confirmed that N-linked glycosylation in both samples only occurred at Asn327. A large number of high mannose glycans, some of which contained xylose and sulfated structures, were detected on antibody from Alg3+ strain; far fewer high mannose N-linked glycans were observed on sample from Alg3− strain. None of the N-linked glycans produced by Alg3− contain xylose, fucose, or galactose. The N-linked glycans produced by Alg3− strain predominantly had a Man3 structure (Table 5).

These analyses show there is a significant difference in the glycan profile after alg3 deletion, With respect to paucimannose N-glycans, based on the method of glycan release, there are between 0 and 3% in the Alg3+ strain profile, while they were more than 89% in the Alg3− strain glycan profile. Similarly, with respect to high mannose N-glycans, based on the method of glycan release, there are between 97% and 100% in the Alg3+ strain profile, while there were less than 11% in the Alg3− strain profile. Thus, the deletion of alg3 resulted in a reduction (up to 90%) in high mannose N-glycans and a simultaneous increase (up to 3000%) in the production of paucimannose N-glycans.
Table 4 below shows N-linked glycans from the alg3+ strain detected by MALDI TOF/TOF MS. Structures were assigned based on ESI− MSⁿ fragmentation of individual peaks. Numerous high mannose (Man5 and higher) core structures are seen.

| Per- | | Cartoon | | |
|---|---|---|---|---|
| methylated | Text description of | representation of | % N-linked glycans³ | |
| mass (m/z)¹ | structures | structures² | PNGaseF | PNGaseA |
| 1171 | Man₃GlcNAc₂ | 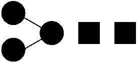 | 2.75 | n.d. |
| 1579 | Man₅GlcNAc₂ or | 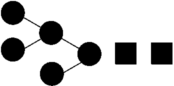 | 12.10 | 11.16 |
| 1168 | Sulph₁Man₅GlcNAc₂ or | 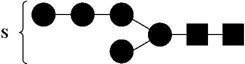 | 5.14 | 7.01 |
| 1740 | Xyl₁Man₅GlcNAc₂ or | 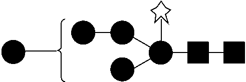 | 3.92 | 2.43 |
| 1783 | Man₆GlcNAc₂ | 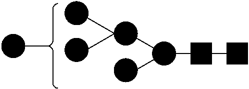 | 23.8 | 25.96 |
| 1872 | Sulph₁Man₆GlcNAc₂ | 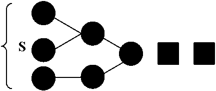 | 5.55 | 10.71 |
| 1987 | Man₇GlcNAc₂ | 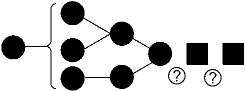 | 17.59 | 16.93 |
| 2033 | Sulph₁Xyl₁Man₆GlcNAc₂ or | 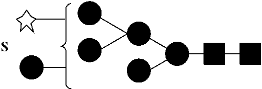 | 2.14 | n.d. |
| 2076 | Sulph₁Man₇GlcNAc₂ | 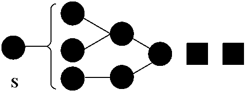 | 3.04 | 5.37 |
| 2191 | Man₈GlcNAc₂ or | 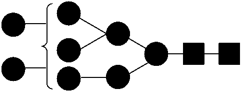 | 10.12 | 8.26 |
| 2234 | Man₇GlcNAc₃ or | 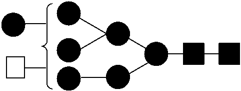 | 4.45 | 3.48 |
| 2395 | Man₉GlcNAc₂ | 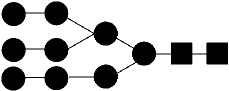 | 7.16 | 6.17 |
| 2438 | Man₈GlcNAc₃ or | 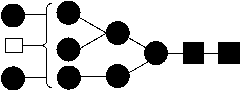 | 2.27 | 1.82 |
| 2642 | Man₉GlcNAc₃ | 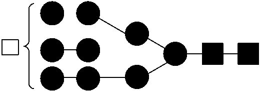 | n.d. | 0.70 |

¹All masses (mass + Na) are single-charged.
²Structures were assigned based on MS¹ mass, MS² fragmentation (CID) and general biosynthetic pathway of N-glycans
³Calculated from the area units of detected N-linked glycan; nd not detected
Legend 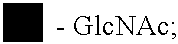 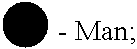 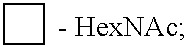 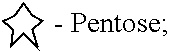
S - Sulfation

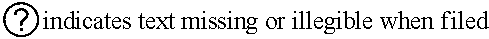

Table 5 below shows N-linked glycans from the alg3− strain detected by MALDI TOF/TOF MS and structures were assigned based on EST− MSⁿ fragmentation of individual peaks.

| | | Cartoon | | |
|---|---|---|---|---|
| Permethylated | Text description of | representation of | % N-linked glycans³ | |
| mass (m/z)¹ | structures | structures² | PNGaseF | PNGaseA |
| 1171 | Man₃GlcNAc₂ | 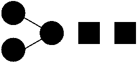 | 40.62 | 40.56 |
| 1260 | Sulph₁Man₃GlcNAc₂ | 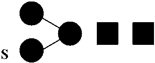 | 40.65 | 39.60 |
| 1375 | Man₄GlcNAc₂ | 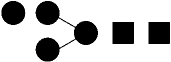 | 8.36 | 8.95 |
| 1579 | Man₅GlcNAc₂ | 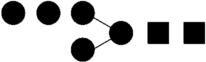 | 7.64 | 7.44 |
| 1783 | Man₆GlcNAc₂ | 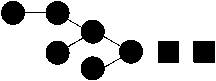 | 2.72 | 2.53 |
| | | 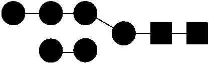 | | |

¹All masses (mass + Na) are single-charged.
²Structures were assigned based on MS¹ mass, MS² fragmentation (CID) and general biosynthetic pathway of N-glycans
³Calculated from the area units of detected N-linked glycans; nd not detected
Legend 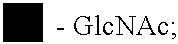 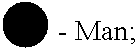
S - Sulfation

Table 6 below shows differences between alg3+ and alg3− strains with respect to high mannose and paucimannose N-glycan profiles. Note that the alg3− strains produced the heterologous glycoprotein in high amounts and free of any xylose, fucose, galactose, or other carbohydrate moieties attached to the Man3NAc2 and/or Man4NAc2 glycan.

| | | % N-linked glycans | |
|---|---|---|---|
| Strain | N-glyans | PNGaseF | PNGaseA |
| #5942 | High | 97 | 100 |
| | mannose | | |
| #5942 | Pauci- | 3 | 0 |
| | mannose | | |
| #6670 | High | 10 | 11 |
| | mannose | | |
| #6670 | Pauci- | 90 | 89 |
| | mannose | | |

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description, and there is no intention in the use of such terms and expressions of excluding any equivalents of the features shown and described or portions thereof, but it is recognized that various modifications are possible within the scope of the invention claimed. In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described as if set forth individually herein. Sub-headings are used for organizational purposes only and to assist the reader, and should not be construed as limiting the disclosure. Other embodiments are within the following claims.

Although the invention has been described with reference to the above examples, it will be understood that modifications and variations are encompassed within the spirit and scope of the invention. Accordingly, the invention is limited only by the following claims.

1. A recombinant cell of the family Thraustochytriaceae for the production of a glycomolecule, comprising
a nucleic acid sequence encoding a heterologous glycomolecule;
a nucleic acid sequence encoding a heterologous oligosaccharyltransferase;
wherein the recombinant cell produces the heterologous glycomolecule having higher glycan occupancy compared to the same heterologous glycomolecule produced by a corresponding cell not comprising the heterologous oligosaccharyltransferase.

2. The recombinant cell of claim 1 wherein the glycomolecule is a glycoprotein or glycopeptide, and wherein the recombinant cell further comprises a genetic modification in a mannosyl transferase gene.

3. The recombinant cell of claim 2 wherein the mannosyl transferase gene is alg3 and the genetic modification is a deletion or disruption.

4. The recombinant cell of claim 3 wherein the heterologous oligosaccharyltransferase is from a protozoa, and further comprises a protozoal promoter that regulates the sequence encoding the heterologous oligosaccharyltransferase.

5. The recombinant cell of claim 4 wherein the heterologous oligosaccharyltransferase is a single protein enzyme.

6. The recombinant cell of claim 3 wherein the oligosaccharyltransferase is from a protozoa of the Family Trypanosomatidae.

7.-8. (canceled)

9. The recombinant cell of claim 5 wherein the protozoal gene comprises a protozoal promoter that regulates the sequence encoding the heterologous oligosaccharyltransferase.

10. The recombinant cell of claim 5 wherein the heterologous oligosaccharyltransferase comprises the Stt3 subunit of a protozoal oligosaccharyltransferase.

11. The recombinant cell of claim 10 wherein the heterologous oligosaccharyltransferase is a protozoal enzyme encoded by a gene selected from the group consisting of: TbStt3A, TbStt3B, LmStt3D, LbStt3_1, and LbStt3_3.

12. (canceled)

13. The recombinant cell of claim 3 wherein the heterologous glycoprotein or glycopeptide has a glycan occupancy of more than 25%.

14. (canceled)

15. The recombinant cell of claim 3 wherein the cell produces and secretes the heterologous glycoprotein or glycopeptide molecule or functional portion thereof.

16. (canceled)

17. The recombinant cell of claim 3 wherein the glycans are N-glycans and comprise Man3-5G1cNAc2.

18. The recombinant cell of claim 3 wherein the heterologous glycoprotein or glycopeptide molecule is an antibody molecule, or portion thereof.

19. The recombinant cell of claim 3 wherein the Thraustochytriaceae cell is of a genus selected from the group consisting of: Japanochytrium, Oblongichytrium, Thraustochytrium, Aurantiochytrium, and Schizochytrium.

20. (canceled)

21. The recombinant cell of claim 3 wherein the heterologous glycoprotein or glycopeptide is selected from the group consisting of: trastuzumab, eculizurnab, natalizurnab, cetuximab, omalizumab, usteinumab, paniturnumab, and adalimurnab, or a functional fragment of any of them.

22. The recombinant cell of claim 4 wherein the heterologous glycoprotein or glycopeptide has a glycan occupancy of more than 25%.

23. (canceled)

24. A composition comprising the heterologous glycoprotein or glycopeptide produced by the recombinant cell of claim 4.

25. The composition of claim 24 wherein the heterologous glycoprotein or glycopeptide is an immunoglobulin.

26.-27. (canceled)

28. A method of producing a glycomolecule, comprising
providing a recombinant Thraustochytriaceae cell comprising a nucleic acid encoding a heterologous glycomolecule;
a sequence encoding a heterologous oligosaccharyltransferase; and
wherein the recombinant cell produces the heterologous glycomolecule having a higher glycan occupancy compared to the same heterologous glycomolecule produced by a corresponding cell not comprising the heterologous oligosaccharyltransferase.

29.-37. (canceled)

38. A recombinant cell of the family Thraustochytriaceae for the production of a glycomolecule, comprising
a nucleic acid sequence encoding a heterologous glycomolecule;
a nucleic acid sequence encoding a promoter that overexpresses an endogenous oligosaccharyltransferase;
wherein the recombinant cell produces the heterologous glycomolecule having higher glycan occupancy compared to the same heterologous glycomolecule produced by a corresponding cell not comprising the heterologous oligosaccharyltransferase.


SEQUENCE LISTING (41 sequences)

SEQ ID NO: 1 - DNA, 3017 bp, Unknown
  Aurantiochytrium sp.
  misc_feature: TEF promoter
agcaggagtggattcggaaggccccaaatggatggcacgagcgagctccttccttcctctcgcgccgcactctctccctccctccctcctctctctcgcgcgcgagtctcgctcactctcctttgcaagagcaacaagcagcctcggcagcgaatgaatgagagtcctccttcgcttctttctcgattcaactcgaagaatgaatgattttcattgctcaaataaataaataaataaataaataaataaataattattgttccattcatggattggcaattacttggttagctagctagctagctagtgagtgagttagtgggttttagtagtgctaacggatggcggcaaagacctcgtcaaaaaaaaatcaagaaagcaagatgaagaagggcctgtgattcaagacccgcgttctgtctcgcttactgcgtggagtgcggagctccgacacgcttgaaattggccaaaagctgcacttcgcgccaccctctgcgccccgaaggtggctttgggccggagcaccaagtttagcgcactgtaaaaaggcgcgaaactttgttggagaagccaattaattaattaattaattaattaatcctttcgacgaaaactaaagaagaagaaagaaatcaagtttccgccctataaaatatcccttcttcacacttccttcattttgtagttagatgataggcagcgaaaggactaaaggtgaaaggcgtagggaccacataggcgcgctagggcggagggaaagatacaaatggcctcagaaaggaagaagaagaggcctcgcggaggaaggatgctgaagcaggaaagatacagcgaaagagaaatcctgtatcttccacagtggatggacaccttcgaggcctgcataagtccacatcactcgctattcaatcattgaattggtcatttaattcaagcatttaattcaatcatgtcttcatgcaatccaccgtccaacaacagagcgcatagaagatgttatccaggtaaggctgcaataatacgcagtttgagttttctattttaaaagtaagtttaaaacttaaaaatttcatacttatgcatgctattcaaaataagattgtatcatcctaaagtattcttcttctcgttcttcttctaatcggaacagagacaactttggtgggtttgcgggcctttgagagaaagaaaaaaaactctcaaaagaaaccaggcttccgaggccgacttgcgcagctctggattgaggttccttcgatcgctcgcttcaccttcctggcccgcgcatgcctcgctctgggtacacagctgagtgagtgagcgaaagatgagcgaatgaatgcaatatttttctattttctattcatttaactgtacttaattaattgattattgattgattgattgattgattgattgattgattaatgactctcgcttctgagaatacatctgttctcatcttcatcgtcacgtcagaatggaaggatgagaaatgaaaagaattcgatcactttcccgccttcttgctagctcatgctcctttcccgccaaaaagaaagaagaggaaagcaccccgaagaaaagaaagaaatcacccaaacaccctcctccttcctcgtccacagacagctcagaataatgaaagctatctttccatcgctcttgacctaactctctttctgctcctgtaaattcatccaacaaatgtttagtctcagaaacccatctgcctcatactactacttactaccttccttacttgaaagcaggcaggctcacggccagcttggcagataggatagttctcatatctattgctgatcgttcccgtttctttctcaaagcaaagtcttttctcttcattccttttctttttcttttcttttcaggctctccacgttttcaggagtagtacatttgctacttagtaattagaaagcttagtactttttgcttttctggattctgaagacttggaaatagaaagaaattaaaaatctttttcttctttctttcagcctttgctggactccctcgcacgcctccttcttccccagccatccatcagcgggcactccacccgcgcttcaacgctcgctcgagtgcgtgcttatttgccttcaacgcggcgcggcggttaatatagtcccagcactccttaaggggggcatcgcagggattatctttttaaaacctgtcacggagttacattttccctcgcatcaaagtgttcccggccgcgtcgcacatctaagttttataacctacacccctcgtggggtaggggcgaattctatgtacacagcacctcagaacttgcgcgcgttccgtgacaaatgaggggtgtggcggcgcattcggccgcatcgccacattcagatatctaacatacccccccttcgcgatgagtggcaggcgaggcggattcgctcgcgagaggcgaggtgccacagcagaccagtaacgaggagccaaggtaggtgaccaccgacgactacgaccacgaccacgaccacagccacggcggctgcagccacgggacgcctcgcatggcagcgcatcagcaccagcaacgacagctgcgaggagcgcagggccgatctggacgcgccggagccgcacgaccaatgccgacgcaacgctgattcttctggattacctctacacatgcatatatgtgtagaggtgcggatgaaatgccctgcgaataaatgaatggcttcgagtttgcctgccgtatgctcgaaagtgcgtgtgcagacacaggcacgaccgagaggacaacagtctgtgcttacctcaccagcacattcttgcaacgccatacgaagcacgcgaaatcttgtggctcagagaggaaggcattcgtgtacgggaacgtggggaacgctatcaatttggaattcaaaatgagtgaaccagacaactaactgtgacttgaactgttgctccacgcatcaaaaccaaacccttaacagaagtagaccagttcgaagctactagcaccaaacaaa

SEQ ID NO: 2 - DNA, 81 bp, Batrachochytrium dendrobatidis
  misc_feature: SP579_nucleotide_sequence
atgccctttaaccgcctttctcttccttgccttcttcttgctctcattgctagcctcttcattcatgctgctcaagctggt

SEQ ID NO: 3 - DNA, 645 bp, Homo sapiens
  misc_feature: trastuzumab_light_chain_nucleotide_sequence
gacatccaaatgacacaaagcccttctagccttagcgcttctgttggtgatcgtgtgaccattacatgtcgtgcttctcaggatgtgaacacagctgttgcttggtaccaacaaaagcctggtaaagctcctaagctcctcatttacagcgctagctttctctactctggcgttccttctcgcttttctggttctagatctggcaccgatttcacactcaccattagctctcttcagcctgaggattttgccacatactactgccagcagcactacacaacacctcctacatttggtcaaggcacaaaggtggagattaagcgtacagttgctgctcctagcgtgttcatttttcctccttctgatgagcagctcaagtctggtacagcttctgttgtttgcctcctcaacaacttctaccctagagaagctaaggtgcagtggaaggttgataacgctcttcaatctggcaactctcaggagtctgttacagagcaagacagcaaggatagcacctactctctttctagcacccttacccttagcaaggctgattacgagaagcacaaggtttacgcttgcgaggttacacatcagggtctttcttctcctgtgaccaagagctttaaccgtggtgaatgttaa

SEQ ID NO: 4 - DNA, 1350 bp, Homo sapiens
  misc_feature: trastuzumab_heavy_chain_nucleotide_sequence
gaggttcagcttgtagaaagtggtggtggtcttgttcaacctggtggttctcttcgtctttcttgtgctgcttctggcttcaacatcaaggatacctacatccactgggttcgtcaagctcctggtaaaggtttagagtgggttgctcgcatttaccctacaaatggctacacacgttacgctgatagcgttaaaggccgctttaccatttctgctgatacctctaagaacaccgcctaccttcagatgaactctcttagagctgaggatacagccgtgtactattgttctagatggggtggtgacggcttttatgctatggattattggggtcagggcacacttgtgacagtttcttctgcttctaccaagggtcctagcgtttttcctttagctccttctagcaagagcacatctggtggtacagctgctttaggttgccttgttaaggactacttccctgaacctgtgacagtttcttggaactctggtgctcttacatctggcgttcacacatttcctgctgttcttcagtcttctggcctctattctcttagctctgtggttacagtgccttcttcttctcttggtacacagacctacatctgcaacgttaaccacaagcctagcaacacaaaggtggacaagaaggttgagcctaagtcttgcgataagacccatacatgtcctccttgtcctgctcctgaattattaggtggtcctagcgtgttcctctttcctcctaaacctaaggacaccctcatgatttctcgcacacctgaagttacatgcgttgtggttgacgtttctcacgaagatcctgaggtgaagttcaactggtacgttgatggtgtggaggttcataacgctaagacaaaacctcgtgaggagcagtacaactctacatatcgcgtggttagcgtgcttacagttcttcatcaggactggcttaacggtaaggagtataagtgcaaggtgagcaacaaggctcttcctgctcctattgagaagaccattagcaaggctaagggccaacctagagaacctcaagtttacacactccctccttctcgtgaagagatgacaaagaaccaggtgtctcttacctgccttgttaagggcttttaccctagcgacattgctgttgaatgggagtctaacggtcaacctgagaacaactacaagacaacacctcctgtgcttgactctgatggcagcttttttctctacagcaagcttaccgtggacaagtctagatggcaacaaggtaacgtgttctcttgctctgtgatgcatgaggctcttcataaccactacacccagaagtctcttagcctttctcctggttaa

SEQ ID NO: 5 - DNA, 4251 bp, Artificial Sequence
  Sythetic
  misc_feature: NLS-Flag-Cas9 (pAM-001)
atgcccaagaaaaagcggaaggtcggcgactacaaggatgacgatgacaagttggagcctggagagaagccctacaaatgccctgagtgcggaaagagcttcagccaatctggagccttgacccggcatcaacgaacgcatacacgagacaagaagtactccatcgggctggacatcgggacgaactccgtgggatgggccgtgatcacagacgaatacaaggtgccttccaagaagttcaaggtgctggggaacacggacagacactccatcaagaagaacctcatcggggccttgctcttcgactccggagaaaccgccgaagcaacgcgattgaaaagaaccgccagaagacgatacacacgacggaagaaccgcatctgctacctccaggagatcttcagcaacgagatggccaaggtggacgactcgttctttcatcgcctggaggagagcttcctggtggaggaagacaagaaacatgagcgccacccgatcttcgggaacatcgtggacgaagtggcctaccacgagaaataccccacgatctaccacttgcgcaagaaactcgtggactccacggacaaagcggacttgcggttgatctacttggccttggcccacatgatcaaatttcggggccacttcctgatcgagggcgacttgaatcccgacaattccgacgtggacaagctcttcatccagctggtgcagacctacaaccagctcttcgaggagaaccccatcaatgcctccggagtggacgccaaagccatcttgtccgcccgattgtccaaatccagacgcttggagaacttgatcgcacaacttcctggcgagaagaagaacggcctcttcggcaacttgatcgcgctgtcgctgggattgacgcctaacttcaagtccaacttcgacttggccgaggacgccaagttgcaactgtccaaggacacctacgacgacgacctcgacaacctgctggcccaaattggcgaccaatacgcggacttgtttttggcggccaagaacttgagcgacgccatcttgttgagcgacatcttgcgcgtgaatacggagatcaccaaagcccctttgtccgcctctatgatcaagcggtacgacgagcaccaccaagacttgaccctgttgaaagccctcgtgcggcaacaattgcccgagaagtacaaggagatcttcttcgaccagtccaagaacgggtacgccggctacatcgacggaggagcctcccaagaagagttctacaagttcatcaagcccatcctggagaagatggacggcaccgaggagttgctcgtgaagctgaaccgcgaagacttgttgcgaaaacagcggacgttcgacaatggcagcatcccccaccaaatccatttgggagagttgcacgccatcttgcgacggcaagaggacttctacccgttcctgaaggacaaccgcgagaaaatcgagaagatcctgacgttcagaatcccctactacgtgggacccttggcccgaggcaattcccggtttgcatggatgacgcgcaaaagcgaagagacgatcaccccctggaacttcgaagaagtggtcgacaaaggagcatccgcacagagcttcatcgagcgaatgacgaacttcgacaagaacctgcccaacgagaaggtgttgcccaagcattcgctgctgtacgagtacttcacggtgtacaacgagctgaccaaggtgaagtacgtgaccgagggcatgcgcaaacccgcgttcctgtcgggagagcaaaagaaggccattgtggacctgctgttcaagaccaaccggaaggtgaccgtgaaacagctgaaagaggactacttcaagaagatcgagtgcttcgactccgtggagatctccggcgtggaggaccgattcaatgcctccttgggaacctaccatgacctcctgaagatcatcaaggacaaggacttcctggacaacgaggagaacgaggacatcctggaggacatcgtgctgaccctgaccctgttcgaggaccgagagatgatcgaggaacggttgaaaacgtacgcccacttgttcgacgacaaggtgatgaagcagctgaaacgccgccgctacaccggatggggacgattgagccgcaaactgattaatggaattcgcgacaagcaatccggaaagaccatcctggacttcctgaagtccgacgggttcgccaaccgcaacttcatgcagctcatccacgacgactccttgaccttcaaggaggacatccagaaggcccaagtgtccggacaaggagactccttgcacgagcacatcgccaatttggccggatcccccgcaatcaaaaaaggcatcttgcaaaccgtgaaagtggtcgacgaactggtgaaggtgatgggacggcacaagcccgagaacatcgtgatcgaaatggcccgcgagaaccaaaccacccaaaaaggacagaagaactcccgagagcgcatgaagcggatcgaagagggcatcaaggagttgggctcccagatcctgaaggagcatcccgtggagaatacccaattgcaaaacgagaagctctacctctactacctccagaacgggcgggacatgtacgtcgaccaagagctggacatcaaccgcctctccgactacgatgtggatcatattgtgccccagagcttcctcaaggacgacagcatcgacaacaaggtcctgacgcgcagcgacaagaaccggggcaagtctgacaatgtgccttccgaagaagtcgtgaagaagatgaagaactactggcggcagctgctcaacgccaagctcatcacccaacggaagttcgacaacctgaccaaggccgagagaggaggattgtccgagttggacaaagccggcttcattaaacgccaactcgtggagacccgccagatcacgaagcacgtggcccaaatcttggactcccggatgaacacgaaatacgacgagaatgacaagctgatccgcgaggtgaaggtgatcacgctgaagtccaagctggtgagcgacttccggaaggacttccagttctacaaggtgcgggagatcaacaactaccatcacgcccatgacgcctacctgaacgccgtggtcggaaccgccctgatcaagaaataccccaagctggagtccgaattcgtgtacggagattacaaggtctacgacgtgcggaagatgatcgcgaagtccgagcaggagatcggcaaagccaccgccaagtacttcttttactccaacatcatgaacttcttcaagaccgagatcacgctcgccaacggcgagatccgcaagcgccccctgatcgagaccaacggcgagacgggagagattgtgtgggacaaaggaagagattttgccacagtgcgcaaggtgctgtccatgcctcaggtgaacatcgtgaagaagaccgaggtgcaaacaggagggttttccaaagagtccattttgcctaagaggaattccgacaagctcatcgcccgcaagaaggactgggaccccaagaagtacgggggcttcgactcccccacggtggcctactccgtgttggtggtggccaaagtggagaaagggaagagcaagaagctgaaatccgtgaaggagttgctcggaatcacgatcatggaacgatcgtcgttcgagaaaaaccccatcgacttcctcgaagccaaagggtacaaagaggtgaagaaggacctgatcatcaagctgcccaagtactccctgttcgagctggagaacggccgcaagcggatgctggcctccgccggggaactgcagaaagggaacgaattggccttgccctccaaatacgtgaacttcctctacttggcctcccattacgaaaagctcaaaggatcccctgaggacaatgagcagaagcaactcttcgtggaacaacacaagcactacctggacgagatcatcgagcagatcagcgagttctccaagcgcgtgatcctcgccgacgccaacctggacaaggtgctctccgcctacaacaagcaccgcgacaagcctatccgcgagcaagccgagaatatcattcacctgtttaccctgacgaatttgggagcccctgccgcctttaaatactttgacaccaccatcgaccgcaaaagatacacctccaccaaggaagtcttggacgccaccctcatccaccagtccatcacgggcctctacgagacgcgcatcgacctctcccaattgggcggcgactaa

SEQ ID NO: 6 - DNA, 788 bp, Streptococcus pyogenes
  misc_feature: hsp60 promoter
acgttcttcgcgaagtcaatccattccccgcgttccccaaatgagggttcgcggtcgaacccgggggctgagaagggccttaaaagcgcgggtttaaagagggatcgggagcggcgggagacaagggattaaggtggaagtggacccttttccagaagggagaaaagcacgagcgggagattgactggtgcagcagatcccgaacgacgtcttcgacaggtacgtgcctcagattgaggtgccgctcatgcggcactgtattcaagcgctctagctggccgccatgttgctgccactctgtttgccgctcgcggccacacggctgccgccaggaccacccaccacccgctccagctgccgtgagctgagcttacctatggacgcatgagcggctccaagccacacgtcctgtctggtgaatatccaacttgacgtcgcggctttgtctccatcattctagctgcgaatctggattgctgaggagatcatcgcttctgcgcggtgtgacgccggcttcagccgcgatagattgatttggatggaagcgaccaagcagagcgtcgcatctccttaccgggtattagggttctgtagatccaaaagacctagtttatgtattgagtggcagagacgaaaaattggctcaggctaatttgaatggctgtggctaagtccttaaatgcttggtggacaatcgatggaagaagagcaaagtgaacaaaaaagactgacctttcaagtttaatttatttgcaatccacaggcgacaaaacaaaacacaaataaaa

SEQ ID NO: 7 - DNA, 21 bp, Artificial Sequence
  Synthetic
  misc_feature: primer oSGI-JU-1360
cttaaatgcttggtggacaat

SEQ ID NO: 8 - protein, 434 aa, Unknown
  Aurantiochytrium sp.
  misc_feature: mannosyl transferase (alg3) SG4EUKT579099
MSLRASKDALVRLRGALDNASTQWWWWAMAATADLALSLLIVKLVPYTEIDFKAYMQEVEGPLLHDEWDYTKLRGDTGPLVYPAGFVYIYMGIRWLTEDGTNLWRGQILFASLHAILVYLVLGSIYYQPDASKDPRRVPFWVGPLAVLSRRVHSIFVLRLFNDGIAMVFMYAAVYMYVRRRWTLGTAFFSAALSVKMNILLFAPGLAVLMLEATGLASSILQAVICVASQIALALPFLQVNAAGYLNRAFELGRVFTYKWTVNFKFLSPEAFVSKALAQGLLSATLLTWVGFGSRHFASSHTGGLRGLVYTSIVRPLKAPLEDTISTVQMHDWKLHVLTLLFTSNFIGIVFARSIHYQFYTWYFHTVSFLVYASGGNFALSLLICVSLEVCFNVYPSTAESSAILQATHLVLLLRLATRKPCPLTAQSKRPKQA

SEQ ID NO: 9 - protein, 434 aa, Unknown; identical to SEQ ID NO: 8
  Aurantiochytrium sp.
  misc_feature: mannosyl transferase (alg3) SG4EUKT579102

SEQ ID NO: 10 - protein, 434 aa, Unknown
  Aurantiochytrium sp.
  misc_feature: mannosyl transferase (alg3) SG4EUKT561246
MSFRASKDALVRLRGALDNASTQWWWWAMAATADLALSLLIVKLVPYTEIDFKAYMQEVEGPLLHDEWDYTKLRGDTGPLVYPAGFVYIYMGIRWLTEDGTNLWRGQILFASLHAILVYLVLGSIYYQPDASKDPRRVPFWVGPLAVLSRRVHSIFVLRLFNDGIAMVFMYAAVYMYVRRRWTLGTAFFSAALSVKMNILLFAPGLAVLMLEATGLASSILQAVICVASQIALALPFLQVNAAGYLNRAFELGRVFTYKWTVNFKFLSPEAFVSKALAQGLLSATLLTWVGFGSRHFASSHTGGLRGLVYTSIVRPLKAPLEDTISTVQMHDWKLHVLTLLFTSNFIGIVFARSIHYQFYTWYFHTVSFLVYASGGNFALSLLICVSLEVCFNVYPSTAESSAILQATHLVLLLRLATRKPCPLTAQSKRPKQA

SEQ ID NO: 11 - DNA, 1305 bp, Unknown
  Aurantiochytrium sp.
  misc_feature: mannosyl transferase SG4EUKT579099
atgtctttgcgtgcgagtaaggatgccctcgtacgtcttcgaggggccctcgacaatgcaagcactcagtggtggtggtgggccatggcagccacggcagacttggcacttagcctgcttattgtgaaactcgtgccttatacggagatcgactttaaagcgtacatgcaagaggttgaaggccccctattgcatgatgaatgggactatacaaagctcaggggcgacacaggcccgctggtttatcctgccggttttgtgtatatttatatgggcatccgctggctcactgaagacggcacgaacctgtggcgaggccagatactttttgcaagtctgcatgcaattcttgtttaccttgtacttggatccatatattaccagccagatgcatcaaaagatcctcgcagagtgccgttctgggtaggacctctagcagtattatcgagacgtgtgcattcaatctttgttctgaggctcttcaacgacggcattgctatggtgtttatgtatgcagcagtatatatgtatgtgcggaggcgttggacgctaggtacggctttcttcagcgcagcacttagcgtgaaaatgaatatactcctatttgcgccaggattagccgtgttgatgctcgaggctacgggtttggcgtcgagcatactgcaggcagtgatctgcgtagcatcacagattgccttagctttgccgttcctccaagtcaacgcagccgggtatctaaatcgggcttttgagctaggtcgtgtctttacgtacaaatggacagtaaacttcaagtttctcagccctgaagcttttgtgagtaaggcacttgcccaaggcctgctgtctgccactttacttacatgggtcggctttgggtctcgccactttgcttcctctcacacaggtggtcttcgcggccttgtgtacacgagcattgttcgaccactgaaagctccgcttgaagacacaatttcaaccgtccaaatgcatgactggaaacttcacgttttgacgctcctattcacaagcaactttattggcatcgtttttgcgcgaagcatccattaccaattctacacttggtactttcacactgtctcattcttagtgtacgccagtggtggaaacttcgcgttgtctcttcttatttgcgtttctctagaagtatgctttaacgtgtatccttcaacagcagaatcgagtgctatcttgcaggcaactcatcttgttttgttattgagacttgctacacgaaaaccttgcccacttacagcacagagcaagcgccctaaacaagcatga

SEQ ID NO: 12 - DNA, 1305 bp, Unknown; identical to SEQ ID NO: 11
  Aurantiochytrium sp.
  misc_feature: mannosyl transferase (alg3) SG4EUKT579102

SEQ ID NO: 13 - DNA, 1305 bp, Unknown
  Aurantiochytrium sp.
  misc_feature: mannosyl transferase (alg3) SG4EUKT561246
atgtctttccgtgcgagtaaggatgccctcgtacgtcttcgaggggccctcgacaatgcaagcactcagtggtggtggtgggccatggcagccacggcagacttggcacttagcctgcttattgtgaaactcgtgccttatacggagatcgactttaaagcgtacatgcaagaggttgaaggccccctactgcatgatgaatgggactatacaaagctcaggggcgacacaggcccgctggtttatcctgctggttttgtgtatatttatatgggcatccgctggctcactgaagacggcacaaacctgtggcgaggccagatactttttgcaagtctgcatgcaattcttgtttaccttgtacttggatccatatactaccagccagatgcatcaaaagatcctcgcagagtgccgttctgggtaggacctctagcagtattatcgagacgtgtgcattcaatctttgttctgaggctcttcaacgacggcattgctatggtgtttatgtatgcagcagtatatatgtatgtgcggaggcgttggacgctaggtacggctttcttcagcgcagcacttagcgtgaaaatgaatatactcctatttgcgccaggattagccgtgttgatgctcgaggctacgggtttggcgtcgagcatactgcaggcagtgatctgcgtagcatcacagattgccttagctttgccgttcctccaagtcaatgcagcagggtatctaaatcgggcttttgagctaggtcgtgtctttacgtacaagtggacagtaaacttcaagtttctcagccctgaagcttttgtaagtaaggcacttgcccaaggcctgctgtctgccactttacttacatgggtcggctttgggtctcgccattttgcttcctctcacacaggtggccttcgcggccttgtgtacacgagcattgttcgaccactgaaagctccgcttgaagacacaatttcaaccgtccaaatgcatgactggaaacttcacgttttgacgctcctattcacaagcaactttattggcatcgtttttgcgcgaagcatccattaccaattctacacttggtactttcacactgtctcattcttagtgtacgccagtggtggaaacttcgcgttgtctcttcttatttgcgtttctctagaagtatgctttaacgtgtatccttcaacagcagaatcgagtgctatcttgcaggcaactcatcttgttttgttattgagacttgctacacgaaaaccttgcccacttacagcacagagcaagcgccctaaacaagcatga

SEQ ID NO: 14 - RNA, 103 nt, Unknown
  Aurantiochytrium sp.
  misc_feature: alg3_gRNA, RNA
gcguacaugcaagagguugaguuuuagagcuagaaauagcaaguuaaaauaaggcuaguccguuaucaacuugaaaaaguggcaccgagucggugcuuuuuuu

SEQ ID NO: 15 - DNA, 120 bp, Artificial Sequence
  Synthetic
  misc_feature: primer oCAB-0341
taatacgactcactatagcgtacatgcaagaggttgagttttagagctagaaatagcaagttaaaataaggctagtccgttatcaacttgaaaaagtggcaccgagtcggtgcttttttt

SEQ ID NO: 16 - DNA, 120 bp, Artificial Sequence
  Synthetic
  misc_feature: primer, oCAB-0342
aaaaaaagcaccgactcggtgccactttttcaagttgataacggactagccttattttaacttgctatttctagctctaaaactcaacctcttgcatgtacgctatagtgagtcgtatta

SEQ ID NO: 17 - DNA, 20 bp, Artificial Sequence
  Synthetic
  misc_feature: primer oJU-0017
cacgacgttgtaaaacgacg

SEQ ID NO: 18 - DNA, 20 bp, Artificial Sequence
  Synthetic
  misc_feature: primer oJU-0001
gttgtgtggaattgtgagcg

SEQ ID NO: 19 - DNA, 30 bp, Artificial Sequence
  Synthetic
  misc_feature: primer oCAB-0294
tactgctctaggattatttattactaggtc

SEQ ID NO: 20 - DNA, 40 bp, Artificial Sequence
  Synthetic
  misc_feature: primer oCAB-0295
cgtcgttttacaacgtcgtgattgcagaattgacgacgtg

SEQ ID NO: 21 - DNA, 40 bp, Artificial Sequence
  Synthetic
  misc_feature: primer oCAB-0296
cgctcacaattccacacaactttatatgggcatccgctgg

SEQ ID NO: 22 - DNA, 27 bp, Artificial Sequence
  Synthetic
  misc_feature: primer oCAB-0297
tacacgttaaagcatacttctagagaa

SEQ ID NO: 23 - DNA, 21 bp, Artificial Sequence
  Synthetic
  misc_feature: primer oCAB-0298
gcttattgtgaaactcgtgcc

SEQ ID NO: 24 - DNA, 21 bp, Artificial Sequence
  Synthetic
  misc_feature: primer oCAB-0299
gcccctgagctttgtatagtc

SEQ ID NO: 25 - DNA, 90 bp, Unknown
  Aurantiochytrium sp.
  misc_feature: SP552
atgaagttcgctacctctgtcgccattgtgcttgttgctaacgttgctaccgctcttgctcagtctgatggttgtacagctaccgatcaa

SEQ ID NO: 26 - DNA, 20 bp, Artificial Sequence
  Synthetic
  misc_feature: primer oSGI-JU-0459
agccacatgcacttcaagag

SEQ ID NO: 27 - DNA, 2457 bp, Unknown
  Aurantiochytrium sp.
  misc_feature: oligosaccharyltransferase ChStt3
atgggcaagaaggccaagtcggctgcccctgtgaagggggcggccagcagcacaacctccgagcctgtggagtcgagtgatgctcccgctgctccccagtacaagctggtggcgaacccaaactcgatgttctggtggggtattcgtatcatcgtgctcgggtttgccattcagctcgcgtacaacatccgactctatgccatcaaagaatacggcctcgtgattcatgagtttgacccctggttcaactaccgcgccaccgagtacctcaaggaccatggtatgcgtgacttctttcgctggtatgaccatatgagctggtatcctctcggacgacctgtcggtacgaccatctaccccggcatgcagatcacctctgtggctatctggaacgctctcgagtcactcggtatgcccatgtcccttaacgacatctgctgctacgtgcccgcctggtttggagtctcggccactatcttcgtaggtcttcttaccgccgagtgcaccggcagccgtaacgctggcgcattcgcatctctggtcatgtcctgcatccctgctcacaccatgcgctccgtcggaggaggctacgacaacgagtccatcgctgttactgccatgagtatgaccttcttcttctggtgccgatccctgcgtgacgacaagtcctggatttttggcattcttactggcctctcttacttctatatggtcgctgcctggggaggatacatctttgtactcaacctcatcggtctgcacgcgattgtcctcgtcgtcaacggtcagttctcccgttctttgtactggtcctacacactcttctacacgattggcactgcttgcgccattcagatccctgtcgtcggcctcacgccgctcaagtctcttgaacagctcggaccttttggagtttggggtatcatgcagcttctctacatctgcgatatcttgcgcgagcgcaggaatctcaatgccaagcagctcttccaggtgcgcatccaggtcttcagcatcgcaggaattgcctttgctgtggtctgcgccatgctctaccccactggatactttggtcctctcagctctcgcgtgcgcagtctctttgtgcagcacacgcgtactggaaacccgctcgtagactctgtggctgagcaccagcccgcgagtgccaacgcttactttcaatacttgcactttgcctgctacctggcgcccatcggtttcatccgctccctcttcagtttgaccaaagctaactcctttttgccgctgtacggagctgtaggatacttcttctcggccaagatggtgcgtcttattatcttgctcggacctatttcatctgcactctccggagttgcattggcgacaatgttggaatggtgctacaaccagttctttatggataaggtcccgctcactccggaggaagtagctgcccaggacaactcttcatccgcgaagaagcgcaagggtgcagctgcccaggaagagccctcggcccttggcccggatatcgaccgtcttatcaagcaagcaaatgtcttctatgagcgcaacggcactgttcgcaagtacgctgctgtcattttgctcatgggtcttggagccatggcaccagagttccacaagtactgccacgctatggcccgtgccatgtcaaacccgagcattatgtacaacgcccgtactcgtgatggccgcactgtgctcgttgatgactaccgcgaggcttacttctggctgcgtgacaacactcctgaggatgctcgtgtaatggcctggtgggactatggctatcagattgctggtattggcaacagaactactattgctgatggaaacacctggaaccacgagcatattgctacccttggtcggtgccttgtgtcccccgaggaaactgcacacaagatgattcgccacctggctgattatgttcttatttggaccggtggtggtggtgatgacctcgccaagatgccgcacattgcgcgtattgctaactctgtgtactcctcggtatgtaatggcgaccctctgtgctcgcagcttggctacattgatcgccagggtacaccctctgagatgatggccaattcgctcatctacaagctccacagtggcttccagcgccccggtgttgttgtagaccagaaccgtttcgagaacgtgttcacttccaagtacaacaaggtacgcatctggcgcgtcaagtctgttgacaaggagtctaaggcctgggctgctgaccttgccaacaagaagtgcgaccctgccccgaacgacttcatctgcaagggtgattacccgccaaagttcagggagttcattaaggatcgccaggactttgctcagcttgaagattttaacgctaagaaaaagaccaaggaggctgaggagtaccagaagcgttaccatgaggagatggctcgtcgtggccagcgtcgcaactaa

SEQ ID NO: 28 - DNA, 2406 bp, Unknown
  Aurantiochytrium sp.
  misc_feature: oligosaccharyltransferase TbStt3A
atgacaaagggcggtaaagtggcagtgacaaagggttctgctcagtctgatggtgctggtgaaggtggtatgtctaaggctaagagctctaccaccttcgtggctacaggtggtggttctttacctgcttgggctcttaaggctgtgagcacaattgtgtctgccgtgattctcatctacagcgttcatcgtgcctacgatattcgccttacctctgttcgcctttacggtgagctcattcacgagtttgacccttggttcaactaccgtgctacccaataccttagcgataacggttggcgcgctttctttcaatggtacgactacatgagctggtaccctcttggtagacctgttggtaccaccatttttcctggcatgcagcttacaggcgtggctattcatcgcgttcttgagatgcttggtcgcggtatgagcatcaacaacatttgcgtgtacatccctgcctggtttggctctattgctactgttcttgctgccctcatcgcttacgagtcatctaacagccttagcgtgatggctttcaccgcttacttcttcagcatcgttcctgctcaccttatgcgctctatggctggtgagtttgacaacgaatgcgtggctatggctgctatgcttctcaccttctacatgtgggttcgctctcttcgctcttcttcttcttggcctattggcgctcttgctggtgttgcttacggttatatggtgtctacatggggcggctacatctttgtgctcaacatggtggctttccacgcttctgtttgcgtgcttcttgattgggctcgcggtatttacagcgtgtctcttcttcgtgcctacagcctctttttcgtgattggtaccgctctcgctatctgtgttcctcctgttgaatggaccccttttcgctctcttgagcagttaaccgctctcttcgtgttcgtgttcatgtgggctcttcactacagcgagtaccttcgtgaacgtgctagagctcctattcacagctctaaggctctccaaattcgtgctcgcatctttatgggcaccctttctcttctcctcatcgttgctagccttcttgctcctttcggcttctttaagcctaccgcttaccgtgttcgcgctctttttgttaagcacacacgcacaggcaaccctcttgttgattctgttgctgagcatcgccctactacagctggtgcttatcttcgctacttccacgtttgctaccctttatggggttgtggtggccttagcatgctcgttttcatgaagaaggaccgttggcgcgctattgtgtttcttgcttctcttagcaccgtgaccatgtacttttctgctcgcatgtctcgccttcttcttttagctggtcctgctgctactgcttgtgctggtatgtttattggcggcctctttgaccttgctcttagccaatttggcgaccttcactctcctaaggacgcttctggtgactctgatcctgctggtggttctaaacgtgctaagggtaaggtggtgaacgagccttctaaacgcgctattttctctcatcgctggtttcaacgcctcgttcagtctttacctgtgcctcttagacgcggtattgctgttgtggttcttgtgtgcctcttcgctaatcctatgcgccactctttcgagaagtcttgcgagaaaatggcccacgctctttcttctcctcgcattattgctgtgaccgaccttcctaatggtgagagagttcttgcagacgactactacgtgagctacctttggcttcgcaacaacacacctgaggatgctcgcattctttcttggtgggattacggttaccagattaccggtatcggtaaccgcacaacactcgctgatggtaatacctggagccacaagcacattgctaccattggcaagatgctcacctctcctgttaaggagtctcacgctcttattcgccaccttgctgattacgtgctcatttgggctggtgaagatcgtggtgacctccttaaatctccccatatggctcgcattggcaactctgtttaccgcgatatgtgctctgaggatgatcctagatgccgccaatttggctttgaaggtggcgatctcaacaagcctacccctatgatgcaacgcagccttctttacaaccttcaccgctttggcacagatggtggtaagacacagctcgataagaacatgttccagctcgcttacgtgagcaagtacggtcttgtgaagatctacaaggtggtgaacgtgtctgaggagtctaaagcttgggtggctgatcctaagaaccgtgtttgtgatcctcctggtagctggatttgtgctggtcaatatcctcctgccaaggagattcaggacatgcttgctaagcgcttccactacgagtag

SEQ ID NO: 29 - DNA, 2466 bp, Unknown
  Aurantiochytrium sp.
  misc_feature: oligosaccharyltransferase TbStt3B
atgacaaagggcggtaaagtggcagtgacaaagggttctgctcagtctgatggtgctggtgaaggtggtatgtctaaggctaagagctctaccaccttcgtggctacaggtggtggttctttacctgcttgggctcttaaggctgtgtctacagtggtttctgccgtgattctcatctactctgttcatcgcgcctacgatattcgccttacatctgttcgcctctacggtgagctcattcacgagtttgatccctggttcaactaccgtgctacccaataccttagcgataacggttggcgcgctttctttcaatggtacgactacatgagctggtaccctcttggtagacctgttggtaccaccatttttcctggcatgcagcttacaggcgtggctattcatcgcgttcttgagatgcttggtcgcggtatgagcatcaacaacatttgcgtgtacatccctgcctggtttggctctattgctactgttcttgctgccctcatcgcttacgagtcatctaacagccttagcgtgatggctttcaccgcttacttcttcagcatcgttcctgctcaccttatgcgctctatggctggtgagtttgacaacgaatgcgtggctatggctgctatgcttctcaccttctacatgtgggttcgctctcttcgctcttcttcttcttggcctattggcgctcttgctggtgttgcttacggttatatggtgtctacatggggcggctacatctttgtgctcaacatggtggctttccacgcttctgtttgcgtgcttcttgattgggctcgtggtacatacagcgtgtctcttcttcgtgcttacagcctcttcttcgtgattggtaccgctctcgctatttgcgttcctcctgttgaatggaccccttttcgctctcttgagcagttaaccgctctcttcgtgttcgtgttcatgtgggctcttcactacagcgagtaccttcgtgaacgtgctagagctcctattcacagctctaaggctctccaaattcgtgctcgcatctttatgggcaccctttctcttctcctcatcgtggctatctacctcttttctaccggctactttcgcccttttagctctagagttcgcgctcttttcgtgaagcatacacgtacaggcaaccctcttgtggattctgttgctgagcatcatcctgctagcaacgacgactttttcggctaccttcacgtgtgctacaacggttggattatcggcttcttcttcatgagcgtgagctgcttcttccactgtacacctggtatgagcttccttctcctctacagcattctcgcctactactttagcctcaagatgtctcgcctccttcttctttctgctcctgttgctagcatccttacaggctacgttgttggcagcattgtggatttagcagctgactgcttcgctgcttctggtacagaacatgctgatagcaaggagcatcagggtaaagctcgtggtaagggtcaaaaggagcagattacagtggagtgcggttgccataaccccttttacaagctttggtgcaacagcttcagctctcgccttgttgttggcaagttctttgtggttgtggtgctcagcatttgcggtcctacctttcttggcagcaactttcgcatctacagcgagcaattcgcagactctatgagctctcctcagatcattatgcgcgctacagttggtggtcgtcgcgttattcttgacgactactacgtgagctacctctggcttcgtaacaacacacctgaggatgctcgcattctttcttggtgggattacggttaccagattaccggtatcggtaaccgcacaacactcgctgatggtaatacctggaaccacgagcacattgctaccattggcaagatgcttaccagccctgttaaggagtctcacgctcttattcgccaccttgctgattacgtgctcatttgggctggctatgatggctctgatctccttaagtctccccatatggctcgcattggcaactctgtttaccgcgatatttgctctgaggacgaccctctttgcacccaatttggcttttactctggcgacttcagcaagcctacccctatgatgcaacgctctcttctctacaaccttcaccgctttggcacagatggtggtaagacacagctcgataagaacatgttccagctcgcttacgtgagcaagtacggtcttgtgaagatctacaaggtgatgaacgtgagcgaggagtctaaagcttgggttgctgatcccaagaaccgtaagtgtgatgctcctggtagctggatttgcacaggtcaatatcctcctgccaaggagattcaggacatgcttgctaagcgcatcgactacgagcaacttgaggactttaaccgtcgtaaccgctctgatgcctactatcgtgcttacatgcgccaaatgggctga

SEQ ID NO: 30 - DNA, 2466 bp, Unknown
  Aurantiochytrium sp.
  misc_feature: oligosaccharyltransferase TbStt3C
atgacaaagggcggtaaagtggcagtgacaaagggttctgctcagtctgatggtgctggtgaaggtggtatgtctaaggctaagagctctaccaccttcgtggctacaggtggtggttctttacctgcttgggctcttaaggctgtgtctacagtggtttctgccgtgattctcatctactctgttcatcgcgcctacgatattcgccttacatctgttcgcctctacggtgagctcattcacgagtttgatccctggttcaactaccgtgctacccaataccttagcgataacggttggcgcgctttctttcaatggtacgactacatgagctggtaccctcttggtagacctgttggtaccaccatttttcctggcatgcagcttacaggcgtggctattcatcgcgttcttgagatgcttggtcgcggtatgagcatcaacaacatttgcgtgtacatccctgcctggtttggctctattgctactgttcttgctgccctcatcgcttacgagtcatctaacagccttagcgtgatggctttcaccgcttacttcttcagcatcgttcctgctcaccttatgcgctctatggctggtgagtttgacaacgaatgcgtggctatggctgctatgcttctcaccttctacatgtgggttcgctctcttcgctcttcttcttcttggcctattggcgctcttgctggtgttgcttacggttatatggtgtctacatggggcggctacatctttgtgctcaacatggtggctttccacgcttctgtttgcgtgcttcttgattgggctcgtggtacatacagcgtgtctcttcttcgtgcttacagcctcttcttcgtgattggtaccgctctcgctatttgcgttcctcctgttgaatggaccccttttcgctctcttgagcagttaaccgctctcttcgtgttcgtgttcatgtgggctcttcactacagcgagtaccttcgtgaacgtgctagagctcctattcacagctctaaggctctccaaattcgtgctcgcatctttatgggcaccctttctcttctcctcatcgtggctatctacctcttttctaccggctactttcgcagctttagctctcgtgttcgcgctcttttcgtgaagcatactcgtacaggcaaccctcttgtggattctgttgctgaacatcgccctacaactgctggtgcttttcttcgtcaccttcacgtttgctacaacggctggattatcggcttcttcttcatgagcgtgagctgcttcttccactgtacacctggtatgagcttccttctcctctacagcattctcgcctactactttagcctcaagatgtctcgcctccttcttctttctgctcctgttgctagcatccttacaggctacgttgttggcagcattgtggatttagcagctgactgcttcgctgcttctggtacagaacatgctgatagcaaggagcatcagggtaaagctcgtggtaagggtcaaaagcgccagattacagttgagtgtggctgccataaccccttctacaaactttggtgcaacagcttcagcagccgcttagttgttggcaagttctttgtggtggtggtgctctctatttgcggtcctacctttcttggcagcgagtttagagctcattgcgagcgttttagcgtgtctgttgcaaatcctcgcatcattagcagcattcgccattctggcaagcttgttcttgcagacgactactacgtgagctacctttggcttcgcaacaacacacctgaggatgctcgcattctttcttggtgggattacggttaccagattaccggtatcggtaaccgcacaacactcgctgatggtaatacctggaaccacgagcacattgctaccattggcaagatgcttaccagccctgttaaggagtctcacgctcttattcgccaccttgctgattacgtgctcatttgggctggtgaagatcgtggtgatcttcgtaagtctcgccatatggctcgcattggtaactctgtttaccgcgatatgtgctctgaggatgaccctctttgcacccaattcggcttttactctggcgacttcaacaagcctacccctatgatgcaacgcagccttctttacaaccttcaccgctttggcacagatggtggtaagacacagctcgataagaacatgttccagctcgcttacgtgagcaagtacggtcttgtgaagatctacaaggtgatgaacgtgagcgaggagtctaaagcttgggttgctgatcccaagaaccgtaagtgtgatgctcctggtagctggatttgtgctggtcaatatcctcctgccaaggagattcaggacatgcttgctaagcgcatcgactacgagcaacttgaggactttaaccgtcgtaaccgctctgatgcctactatcgtgcttacatgcgccaaatgggctga

SEQ ID NO: 31 - DNA, 2574 bp, Unknown
  Aurantiochytrium sp.
  misc_feature: oligosaccharyltransferase LmStt3D
atgggtaagcgtaaggggaatagtcttggcgattctggttctgctgctacagcttctcgtgaagcttctgctcaagctgaagatgctgcttctcagacaaagaccgcttctcctcctgctaaggtgattcttctccctaagacccttaccgacgagaaggactttatcggcatcttccctttccccttttggcctgttcactttgtgcttacagtggtggctctctttgtgttagctgctagctgctttcaggcctttactgttcgcatgatcagcgtgcagatctacggttacctcattcacgagttcgacccttggttcaactacagagctgctgagtacatgagcacacatggttggagcgctttctttagctggttcgactacatgagctggtaccctcttggtagacctgttggttctaccacataccctggccttcagttaacagctgtggctattcatcgcgctcttgctgctgctggtatgcctatgtctctcaacaacgtttgcgtgctcatgcctgcttggtttggtgctattgctacagctaccctcgctttttgcacctatgaggcttctggctctacagttgctgcagccgcagctgctctttctttcagcattatccctgctcacctcatgcgctctatggctggtgaatttgacaacgagtgcatcgctgttgctgctatgcttcttaccttctactgctgggttcgctctcttagaacacgctcttcttggcctattggcgtgttaacaggcgttgcttacggctatatggctgctgcttggggtggttacatctttgtgctcaacatggtggccatgcatgctggtattagctctatggtggactgggctcgtaacacatacaaccctagccttcttcgcgcttacaccctcttttacgttgttggtaccgctatcgctgtgtgtgttcctcctgttggtatgagccctttcaagtctcttgagcaacttggcgctcttctcgttctcgtgtttctttgtggccttcaggtttgcgaggttcttagagctagagctggtgttgaggttcgttctcgtgcaaacttcaagattcgcgttcgcgtgtttagcgtgatggctggtgttgctgctctcgctatttctgttcttgctcctacaggctacttcggtcctctttctgttcgtgttcgcgctctttttgtggagcatactcgtacaggcaaccctcttgtggattctgttgctgagcaccaacctgcttctcctgaagctatgtgggcttttcttcacgtttgcggcgttacatggggtttaggctctattgtgctcgctgtgagcacctttgtgcactattctcctagcaaggtgttctggctcctcaattctggtgctgtgtactacttctctacccgtatggctcgccttcttcttctttctggtcctgctgcttgcctttctacaggcatttttgtgggcaccatccttgaagctgctgttcaactcagcttctgggactctgatgctacaaaggccaagaagcagcagaagcaagctcaacgtcatcaaagaggtgctggtaagggttctggtcgtgatgatgctaagaacgctacaacagctcgcgctttttgcgacgtttttgctggtagcagccttgcttggggtcatagaatggtgctcagcattgctatgtgggctcttgtgacaacaaccgctgtgagcttcttttctagcgagtttgctagccacagcaccaagtttgctgagcaatctagcaaccccatgatcgtgtttgctgctgttgtgcaaaaccgcgctactggtaaacccatgaaccttcttgtggacgactaccttaaggcctacgaatggcttcgcgattctacacctgaggatgctcgtgttcttgcttggtgggattacggttaccagattaccggtatcggtaaccgcacctctcttgctgatggtaatacctggaaccacgagcacattgctaccattggcaagatgcttaccagccctgttgttgaagctcactctcttgttcgccacatggctgattacgtgctcatttgggctggccaatctggtgatctcatgaaatctccccacatggctcgcattggcaactctgtttaccacgacatttgccctgatgaccctctttgccagcaatttggctttcatcgcaacgactactctcgccctacacctatgatgagagctagccttctctacaacctccatgaagctggtaagcgtaagggcgttaaggttaaccctagcctctttcaggaggtgtacagctctaagtacggccttgttcgcatcttcaaggtgatgaacgtgtctgctgagagcaagaagtgggttgctgatcctgctaatcgcgtttgtcatcctcctggtagctggatttgtcctggtcaatatcctcctgccaaggagattcaggagatgttagctcatcgcgtgccttttgatcaggtgacaaacgctgatcgcaagaacaacgttggctcttaccaagaggagtacatgcgtcgtatgcgtgaatctgagaaccgccgttaa

SEQ ID NO: 32 - DNA, 2319 bp, Unknown
  Aurantiochytrium sp.
  misc_feature: oligosaccharyltransferase LbStt3_1
atgtactgcctcaacaaagcctaccgcattcgcatgtttagcgtgcaactttacggctacatcatccacgagttcgatccttggttcaactacagagctgccgagtacatgtctgctcatggttggtctgccttctttagctggttcgactacatgagctggtaccctcttggtagacctgttggtacaaccacataccctggccttcagttaacagctgtggctattcatcgcgctcttgctgctgctggtgttcctatgtctctcaacaacgtttgcgtgctcatccctgcttggtatggtgctattgctaccgctcttgaggctctcatgatctatgagtgcaacggtagcggcattacagctgctattggcgctttcatcttcatgatcctccctgctcaccttatgcgctctatggctggtgagtttgacaacgagtgcattgctgttgctgccatgcttcttaccttctacctttgggttcgctctcttcgtacacgttgttcttggcctatcggcattcttaccggcattgcttacggctacatggttgctgcttggggtggttacatctttgtgctcaacatggtggccatgcatgctggtattagctctatggtggactgggctcgtaacacatacaaccctagccttcttcgcgcttacgctctcttttacgttgttggtaccgctatcgctacacgtgttcctcctgttggtatgagcccttttcgctctcttgagcaacttggtgctcttgctgtgctcctttttctttgcggccttcaagcttgcgaggtttttagagctcgcgctgatgttgaggttcgttctagagctaacttcaagatccgcatgcgcgcttttagcgttatggctggtgttggtgctctcgctattgctgttctttctcctacaggctacttcggtcctcttacagctagagttcgcgctctctttatggagcatacacgtacaggcaaccctcttgtggattctgttgctgaacaccgcaagacaaaccctcaagcttacgagtacttcctcgacttcacctacagcatgtggatgttaggcgctgttctccagcttttaggtgctgctgttggttctcgtaaggaagctcgcctctttatgggcctttactctcttgctacctactacttcagcgaccgcatgtctcgtcttatggttttagctggtcctgctgctgctgctattgctgctgaaattctcggcatcccttacgaatggtgctggacacaacttacaggttgggcttctccaaacacctctgctagagaacgcaagtctaaggaggatggtccttgcaagacaaagcgcaatcaacgccagacagtggctacaaagcttgatcatggtgctagagctcgcgctacagctgctgttaagtttatggagaccgctcttgagcgcgttcctcttgtttttagagctgctatcgccatcggcatcattggtgctacagttggtaccccttacgtgtaccagtttcaagctcgctgcattcagagctcttacagctttgctgttccccgcatcatgtttcacacccaacttagaactggcgagaccgtgattgtgaaggattacgttgaggcctacgagtggcttcgtgataatacacctgcagatgctcgcgtgctttcttggtgggattatggttaccagatcaccggtatcggtaaccgtacctctcttgctgatggcaatacctggaaccacgagcatattgctaccattggcaagatgctcacctctcctgttgctgaagctcactctcttgttcgccatatggctgattacgtgctcatttgggctggtcaaggtggtgatctcatgaaatctccccacatggctcgcattggcaactctgtttaccacgacatttgcccaaacgaccctctttgccagcattttggcttctacgaggactactctcgccctaaacccatgatgagagctagccttctctacaacctccatgaagctggtcgttctgctggtgttaaggttgatcctagcctctttcaggaggtgtacagctctaagtacggccttgttcgcatcttcaaggtgatgaacgtgtctgctgagagcaagaagtgggttgctgatcctgctaatcgcgtttgtcatcctcctggtagctggatttgtcctggtcaatatcctcctgccaaggagattcaggagatgttagctcatcgcgtgccttttgaccaaatgggtaagaagcacgacgacacacacaaagctcgtatggctagatctcgcacacttggtgaggcttaa

SEQ ID NO: 33 - DNA, 2565 bp, Unknown
  Aurantiochytrium sp.
  misc_feature: oligosaccharyltransferase LbStt3_3
atgggcaagaagaaggcaattccgtctggctctgttggtcctgctacaacaacatctcgtgaagctcctggcaaggatgaaggtgcttctcaacctgctaagacagctgctcttcctgttaagcctttcgtgcttccaaacacccttaccgatgaagaggagtttgtgggcatctttccttgccctttttggcctgttcgcttcgtgattaccgtgatggctcttgtgcttcttggcgcttcttgcattcgcgctttcacaattcgcatgcttagcgttcagctctacggctacatcattcacgagtttgacccctggttcaactacagagctgctgaatacatgagcgctcatggttggagcgctttctttagctggttcgactacatgagctggtaccctcttggtagacctgttggtacaaccacataccctggccttcagttaacagctgtggctattcatcgcgctcttgctgctgctggtgttcctatgtctctcaacaacgtttgcgtgctcatccctgcttggtatggtgctattgctaccgctattctcgctctttgcgcttacgaggtttctcgttctatggttgctgctgctgttgctgctctctctttcagcattatccctgctcaccttatgcgctctatggctggtgagtttgacaacgagtgcattgctgttgctgccatgcttcttaccttctacctttgggttcgctctcttcgtacacgttgttcttggcctatcggcattcttaccggcattgcttacggctacatggttgctgcttggggtggttacatctttgtgctcaacatggtggccatgcatgctggtattagctctatggtggactgggctcgtaacacatacaaccctagccttcttcgcgcttacgctctcttttacgttgttggtaccgctatcgctacacgtgttcctcctgttggtatgagcccttttcgctctcttgagcaacttggtgctcttgctgtgctcctttttctttgcggccttcaagcttgcgaggtttttagagctcgcgctgatgttgaggttcgttctagagctaacttcaagatccgcatgcgcgcttttagcgttatggctggtgttggtgctctcgctattgctgttctttctcctacaggctacttcggtcctcttacagctagagttcgcgctctctttatggagcatacacgtacaggcaaccctcttgtggattctgttgctgagcatcatcctgcttctcctgaagctatgtggacctttcttcacgtttgcggcgttacatggggtttaggctctattgtgctccttgtgtctctccttgtggactactctagcgctaagctcttttggctcatgaactctggcgctgtgtactacttttctacccgcatgtctcgccttcttcttcttacaggtcctgctgcttgcctttctacaggttgctttgttggcacccttcttgaagctgctatccagttcaccttctggagctctgatgctacaaaggccaagaagcaacaggagacacagcttcatcagaagggtgctcgtaagcactctgatcgctcaaacagcaagaacgctcttacagttcgcacacttggcgatgttcttcgctctacaagcttagcttggggtcatcgtatggtgctttgctttgctatgtgggctctcgtgattacagtggctgtttgccttcttggcagcgattttacctctcacgctaccatgtttgctcgccaaacatctaaccccctcatcgtttttgccaccgttcttagagatcgcgctacaggtaagcctacacaggttcttgtggacgactaccttcgctcttacctttggcttcgcgataacacacctcgtaatgctcgcgttctttcttggtgggattacggttaccagattaccggtatcggtaaccgcacctctcttgctgatggtaatacctggaaccacgagcacattgctaccattggcaagatgcttaccagccctgttgctgaagctcactctcttgttcgccatatggctgattacgtgctcatttgggctggtcaaggtggtgatctcatgaaatctccccacatggctcgcattggcaactctgtttaccacgacatttgcccaaacgaccctctttgccagcattttggcttctacaagaacgaccgcaaccgtcctaagcctatgatgagagctagcctcctctacaaccttcatgaagctggtcgttctgctggcgttaaggttgatcctagcctctttcaggaggtgtacagctctaagtacggccttgttcgcatcttcaaggtgatgaacgtgtctgctgagagcaagaagtgggttgctgatcctgctaatcgcgtttgtcatcctcctggtagctggatttgtcctggtcaatatcctcctgccaaggagattcaggagatgttagctcatcgcgtgccttttgatcacgtgaacagctttagccgcaagaaagctggctcttaccatgaggagtacatgagacgtatgcgtgaggagcaagatcgctaa

SEQ ID NO: 34 - protein, 846 aa, Unknown
  Aurantiochytrium sp.
  misc_feature: oligosaccharyltransferase ChStt3
MKNKQLKQQTANTYRHQSANGDSLTRAKMGKKAKSAAPVKGAASSTTSEPVESSDAPAAPQYKLVANPNSMFWWGIRIIVLGFAIQLAYNIRLYAIKEYGLVIHEFDPWFNYRATEYLKDHGMRDFFRWYDHMSWYPLGRPVGTTIYPGMQITSVAIWNALESLGMPMSLNDICCYVPAWFGVSATIFVGLLTAECTGSRNAGAFASLVMSCIPAHTMRSVGGGYDNESIAVTAMSMTFFFWCRSLRDDKSWIFGILTGLSYFYMVAAWGGYIFVLNLIGLHAIVLVVNGQFSRSLYWSYTLFYTIGTACAIQIPVVGLTPLKSLEQLGPFGVWGIMQLLYICDILRERRNLNAKQLFQVRIQVFSIAGIAFAVVCAMLYPTGYFGPLSSRVRSLFVQHTRTGNPLVDSVAEHQPASANAYFQYLHFACYLAPIGFIRSLFSLTKANSFLPLYGAVGYFFSAKMVRLIILLGPISSALSGVALATMLEWCYNQFFMDKVPLTPEEVAAQDNSSSAKKRKGAAAQEEPSALGPDIDRLIKQANVFYERNGTVRKYAAVILLMGLGAMAPEFHKYCHAMARAMSNPSIMYNARTRDGRTVLVDDYREAYFWLRDNTPEDARVMAWWDYGYQIAGIGNRTTIADGNTWNHEHIATLGRCLVSPEETAHKMIRHLADYVLIWTGGGGDDLAKMPHIARIANSVYSSVCNGDPLCSQLGYIDRQGTPSEMMANSLIYKLHSGFQRPGVVVDQNRFENVFTSKYNKVRIWRVKSVDKESKAWAADLANKKCDPAPNDFICKGDYPPKFREFIKDRQDFAQLEDFNAKKKTKEAEEYQKRYHEEMARRGQRRN

SEQ ID NO: 35 - protein, 801 aa, Trypanosoma brucei
  misc_feature: oligosaccharyltransferase TbStt3A
MTKGGKVAVTKGSAQSDGAGEGGMSKAKSSTTFVATGGGSLPAWALKAVSTIVSAVILIYSVHRAYDIRLTSVRLYGELIHEFDPWFNYRATQYLSDNGWRAFFQWYDYMSWYPLGRPVGTTIFPGMQLTGVAIHRVLEMLGRGMSINNICVYIPAWFGSIATVLAALIAYESSNSLSVMAFTAYFFSIVPAHLMRSMAGEFDNECVAMAAMLLTFYMWVRSLRSSSSWPIGALAGVAYGYMVSTWGGYIFVLNMVAFHASVCVLLDWARGIYSVSLLRAYSLFFVIGTALAICVPPVEWTPFRSLEQLTALFVFVFMWALHYSEYLRERARAPIHSSKALQIRARIFMGTLSLLLIVASLLAPFGFFKPTAYRVRALFVKHTRTGNPLVDSVAEHRPTTAGAYLRYFHVCYPLWGCGGLSMLVFMKKDRWRAIVFLASLSTVTMYFSARMSRLLLLAGPAATACAGMFIGGLFDLALSQFGDLHSPKDASGDSDPAGGSKRAKGKVVNEPSKRAIFSHRWFQRLVQSLPVPLRRGIAVVVLVCLFANPMRHSFEKSCEKMAHALSSPRIIAVTDLPNGERVLADDYYVSYLWLRNNTPEDARILSWWDYGYQITGIGNRTTLADGNTWSHKHIATIGKMLTSPVKESHALIRHLADYVLIWAGEDRGDLLKSPHMARIGNSVYRDMCSEDDPRCRQFGFEGGDLNKPTPMMQRSLLYNLHRFGTDGGKTQLDKNMFQLAYVSKYGLVKIYKVVNVSEESKAWVADPKNRVCDPPGSWICAGQYPPAKEIQDMLAKRFHYE

SEQ ID NO: 36 - protein, 801 aa, Trypanosoma brucei; identical to SEQ ID NO: 35
  misc_feature: oligosaccharyltransferase TbStt3B

SEQ ID NO: 37 - protein, 821 aa, Trypanosoma brucei
  misc_feature: oligosaccharyltransferase TbStt3C
MTKGGKVAVTKGSAQSDGAGEGGMSKAKSSTTFVATGGGSLPAWALKAVSTVVSAVILIYSVHRAYDIRLTSVRLYGELIHEFDPWFNYRATQYLSDNGWRAFFQWYDYMSWYPLGRPVGTTIFPGMQLTGVAIHRVLEMLGRGMSINNICVYIPAWFGSIATVLAALIAYESSNSLSVMAFTAYFFSIVPAHLMRSMAGEFDNECVAMAAMLLTFYMWVRSLRSSSSWPIGALAGVAYGYMVSTWGGYIFVLNMVAFHASVCVLLDWARGTYSVSLLRAYSLFFVIGTALAICVPPVEWTPFRSLEQLTALFVFVFMWALHYSEYLRERARAPIHSSKALQIRARIFMGTLSLLLIVAIYLFSTGYFRSFSSRVRALFVKHTRTGNPLVDSVAEHRPTTAGAFLRHLHVCYNGWIIGFFFMSVSCFFHCTPGMSFLLLYSILAYYFSLKMSRLLLLSAPVASILTGYVVGSIVDLAADCFAASGTEHADSKEHQGKARGKGQKRQITVECGCHNPFYKLWCNSFSSRLVVGKFFVVVVLSICGPTFLGSEFRAHCERFSVSVANPRIISSIRHSGKLVLADDYYVSYLWLRNNTPEDARILSWWDYGYQITGIGNRTTLADGNTWNHEHIATIGKMLTSPVKESHALIRHLADYVLIWAGEDRGDLRKSRHMARIGNSVYRDMCSEDDPLCTQFGFYSGDFNKPTPMMQRSLLYNLHRFGTDGGKTQLDKNMFQLAYVSKYGLVKIYKVMNVSEESKAWVADPKNRKCDAPGSWICAGQYPPAKEIQDMLAKRIDYEQLEDFNRRNRSDAYYRAYMRQMG

SEQ ID NO: 38 - protein, 857 aa, Leishmania major
  misc_feature: oligosaccharyltransferase LmStt3D
MGKRKGNSLGDSGSAATASREASAQAEDAASQTKTASPPAKVILLPKTLTDEKDFIGIFPFPFWPVHFVLTVVALFVLAASCFQAFTVRMISVQIYGYLIHEFDPWFNYRAAEYMSTHGWSAFFSWFDYMSWYPLGRPVGSTTYPGLQLTAVAIHRALAAAGMPMSLNNVCVLMPAWFGAIATATLAFCTYEASGSTVAAAAAALSFSIIPAHLMRSMAGEFDNECIAVAAMLLTFYCWVRSLRTRSSWPIGVLTGVAYGYMAAAWGGYIFVLNMVAMHAGISSMVDWARNTYNPSLLRAYTLFYVVGTAIAVCVPPVGMSPFKSLEQLGALLVLVFLCGLQVCEVLRARAGVEVRSRANFKIRVRVFSVMAGVAALAISVLAPTGYFGPLSVRVRALFVEHTRTGNPLVDSVAEHQPASPEAMWAFLHVCGVTWGLGSIVLAVSTFVHYSPSKVFWLLNSGAVYYFSTRMARLLLLSGPAACLSTGIFVGTILEAAVQLSFWDSDATKAKKQQKQAQRHQRGAGKGSGRDDAKNATTARAFCDVFAGSSLAWGHRMVLSIAMWALVTTTAVSFFSSEFASHSTKFAEQSSNPMIVFAAVVQNRATGKPMNLLVDDYLKAYEWLRDSTPEDARVLAWWDYGYQITGIGNRTSLADGNTWNHEHIATIGKMLTSPVVEAHSLVRHMADYVLIWAGQSGDLMKSPHMARIGNSVYHDICPDDPLCQQFGFHRNDYSRPTPMMRASLLYNLHEAGKRKGVKVNPSLFQEVYSSKYGLVRIFKVMNVSAESKKWVADPANRVCHPPGSWICPGQYPPAKEIQEMLAHRVPFDQVTNADRKNNVGSYQEEYMRRMRESENRR

SEQ ID NO: 39 - protein, 772 aa, Leishmania brasiliensis
  misc_feature: oligosaccharyltransferase LbStt3_1
MYCLNKAYRIRMFSVQLYGYIIHEFDPWFNYRAAEYMSAHGWSAFFSWFDYMSWYPLGRPVGTTTYPGLQLTAVAIHRALAAAGVPMSLNNVCVLIPAWYGAIATALEALMIYECNGSGITAAIGAFIFMILPAHLMRSMAGEFDNECIAVAAMLLTFYLWVRSLRTRCSWPIGILTGIAYGYMVAAWGGYIFVLNMVAMHAGISSMVDWARNTYNPSLLRAYALFYVVGTAIATRVPPVGMSPFRSLEQLGALAVLLFLCGLQACEVFRARADVEVRSRANFKIRMRAFSVMAGVGALAIAVLSPTGYFGPLTARVRALFMEHTRTGNPLVDSVAEHRKTNPQAYEYFLDFTYSMWMLGAVLQLLGAAVGSRKEARLFMGLYSLATYYFSDRMSRLMVLAGPAAAAIAAEILGIPYEWCWTQLTGWASPNTSARERKSKEDGPCKTKRNQRQTVATKLDHGARARATAAVKFMETALERVPLVFRAAIAIGIIGATVGTPYVYQFQARCIQSSYSFAVPRIMFHTQLRTGETVIVKDYVEAYEWLRDNTPADARVLSWWDYGYQITGIGNRTSLADGNTWNHEHIATIGKMLTSPVAEAHSLVRHMADYVLIWAGQGGDLMKSPHMARIGNSVYHDICPNDPLCQHFGFYEDYSRPKPMMRASLLYNLHEAGRSAGVKVDPSLFQEVYSSKYGLVRIFKVMNVSAESKKWVADPANRVCHPPGSWICPGQYPPAKEIQEMLAHRVPFDQMGKKHDDTHKARMARSRTLGEA

SEQ ID NO: 40 - protein, 854 aa, Leishmania brasiliensis
  misc_feature: oligosaccharyltransferase LbStt3_3
MGKKKAIPSGSVGPATTTSREAPGKDEGASQPAKTAALPVKPFVLPNTLTDEEEFVGIFPCPFWPVRFVITVMALVLLGASCIRAFTIRMLSVQLYGYIIHEFDPWFNYRAAEYMSAHGWSAFFSWFDYMSWYPLGRPVGTTTYPGLQLTAVAIHRALAAAGVPMSLNNVCVLIPAWYGAIATAILALCAYEVSRSMVAAAVAALSFSIIPAHLMRSMAGEFDNECIAVAAMLLTFYLWVRSLRTRCSWPIGILTGIAYGYMVAAWGGYIFVLNMVAMHAGISSMVDWARNTYNPSLLRAYALFYVVGTAIATRVPPVGMSPFRSLEQLGALAVLLFLCGLQACEVFRARADVEVRSRANFKIRMRAFSVMAGVGALAIAVLSPTGYFGPLTARVRALFMEHTRTGNPLVDSVAEHHPASPEAMWTFLHVCGVTWGLGSIVLLVSLLVDYSSAKLFWLMNSGAVYYFSTRMSRLLLLTGPAACLSTGCFVGTLLEAAIQFTFWSSDATKAKKQQETQLHQKGARKHSDRSNSKNALTVRTLGDVLRSTSLAWGHRMVLCFAMWALVITVAVCLLGSDFTSHATMFARQTSNPLIVFATVLRDRATGKPTQVLVDDYLRSYLWLRDNTPRNARVLSWWDYGYQITGIGNRTSLADGNTWNHEHIATIGKMLTSPVAEAHSLVRHMADYVLIWAGQGGDLMKSPHMARIGNSVYHDICPNDPLCQHFGFYKNDRNRPKPMMRASLLYNLHEAGRSAGVKVDPSLFQEVYSSKYGLVRIFKVMNVSAESKKWVADPANRVCHPPGSWICPGQYPPAKEIQEMLAHRVPFDHVNSFSRKKAGSYHEEYMRRMREEQDR

SEQ ID NO: 41 - DNA, 3000 bp, Unknown
  Aurantiochytrium sp.
  misc_feature: actin promoter
ttcatctataaagtttgatgaagattagttcaaagatcgacaatgggaagtctaggtagttatggactaccataagacacctagcttctgtgatgcatcggggaaatgcatcggcactggaccttgtggttgccaagccgtcaagtcaaaagtgtggactagacttcaaaaactctctttattcaagatactcacaattgaaaccaatgcttggagaattgttggacacctagcctcgaatgaatttccgtaacttatgaacaaatttgatgatgctttttgtatcaatatgctaaatgaaatggtcaaaaggtctggaatactttacaatgtccatcaggtctctaattcgtaagtcgaaactcctttgcctttatttgtcactgtactcatgactaaacttctatatctttactaatcgactttccagtctataaatgtcaattattagtgctaccaagagaattttttttgatatgatgctacccccaatacgtagacgaggcaactccttagatcttttagctagagtagaagcctcaatccaaatcaagcacttgtatgatgcagaagcctttcaagcaatattcttgtcgaaaagaaacttgcagcgtctgaaaggtcgttgacaggtcgcgaacacccgtactactgctgtgagcttcgaaagaccttttcacacatcttaagatgtgaaatgtagggcctattctctctcttttgctaaactttcacttcatgcctaaagttactatctatgtacctagccaggtacctacataccacccatgcaatgagtgattggtttggctagagtgtttaagaattgacatgagttgcagtgagagtacaacttttcattttatatgatacaatacacaagattatatcaccgtgatgatggtcgctagatgttggctcacacacactcactaggaacctggtcatagcgtttgattggcgagtgtgtgtttactcaggatttctatccctgcaaacgaattgaaaagtacaaacaagtcaaaacaaagaggaccctatttgatgcaaagttgtgtattttcaatcaaagagacttgaaagagtagagtgagttgcaatccgtcttttggaagcagacaacatgctggcaagcttaaagcaaagcatacttgaagcaaatcttaacttgaacgcgggagtgttgcgatcatggatgcaataaaaatccaaaggttagggttttcaaagttagtatactgtcggctgactcgttctgtgtatttaaagcccccctccccccctctgatgggattctgtccttatttaattaaatggacacacccaacttgaactttagcataatcgtggctttttaattgtaaaacgtaactaaatgtcgtgctaacacactgcgcacgactcacacaagtcaaaggctattcctacaaattacattcttcttgatagaacttaaggaaaatcttattgctctaccatagcatgggttcgggaaaactttaaaagaaaaaagcatactcttcatactctcgtatttcctaatttatttgagcacgagccaacacaagctccctcgtaagagaaggtagtaggtactttagcagtgagcatctgggtagaggtatctgccttctaatatcacctacctcaaggtccgtgccacgcgcgagggaaactctgaagaagactaggaagtgcactactactccacgagggaatcccgctttcacgagatactcaactacattctcagccagtaggcacccagcactctgtagtagctgtacctaatggaagactagatgctctgtacactcaacttacctacttctgtttctgcggtgtagaatatcgcagtcattaactaaaaacaagataaaaatgagaatactttgtaagtttaatttattattagtagcaatcatatcatatatggaatctttttcgaaagataaagcaaaaaataaacattattttggaaataaaaagaattgttaatcaaagcgtaagacgtcctatacagctgactgtatgatgggacattaggtaaattggtcctaagaaggttcacccaagtcattgaccattcaagttgagtaaagctagtgattcaagttgttttgacttgagttttttacccaagttaaaagatctcaactcagtacctctgactacctcgtgagagtggccattggcttttgatatttacttgtgtaagaagagttcctcccgacggcaggtgggcagtagtacctaccaataggagaagcgctacgtgctattcctgaagtacacaccacgtaggtgagtgagttttattattctttttattttaaacataatgtgtatgaagcttactatagttagttaattttagaaataccataccataatatatcatctttatatagtcggggtacaacagaaaagggcaatgaaaatcgactttgggcgggcgagtgagagtccgcagctgctctggccttcgggtcggtgtccgcactcacattggtagtctgtagacagaatttggaccttctgtaggcagagagtacctactaggagcgtcttccaataatcgcctcgatttccccaacctggatgatgctggtggctcaacttgaactaaaacctgaggatgaaggagccactcgattccacgcacacccttcaggtggtcatttgcaggttagcgatagaggtatctccctcacaatacactgtaaatagttttgtgattaaatacacacacgagcactcctataaagggtgtgtaagcaaaggaaattcctctcacaacacactgagtatcaaaagaggaacctaggactaagaaggttatcatagatggatctaatcagaggaggtaacactgtaaatttgtggagacagtggagggtctttggccacgaagatctgcaagcgcgccatcagcagatccgcaaccttcgagctcaagaagcaactcaacagtagaagaacaagcacccaactagcaaa